(19) United States
(12) Patent Application Publication
(10) Pub. No.: US 2004/0033555 A1
(43) Pub. Date: Feb. 19, 2004

(54) Methods using light emission for determining the effeciveness of plant treatment agents in controlling plant disease organisms

(76) Inventors: Shawn Louise Anderson, West Grove, PA (US); Kathleen Ann Lu, Newark, DE (US)

Correspondence Address: David E Heiser, E I du Pont de Nemours and Company, Legal-Patents, Wilimington, DE, 19898, US

(21) Appl. No.: 10/343,073

(22) PCT Filed: Jul. 20, 2001
(86) PCT No.: PCT/US01/22932

(51) Int. Cl. C12Q 1/04

(52) U.S. Cl. 435/34

(57) ABSTRACT

Methods are provided for determining the effectiveness of a plant treatment agent in controlling a plant disease organism. The methods involve (a) applying the plant treatment agent to a substrate, (b) inoculating the substrate with the disease organism, and detecting photon emission from light-generating moieties. The methods also involve (1c) after a period of time in which the disease organism can grow, applying a pro-light-generating moiety which is selectively transformed by the disease organism or the substrate to a light-generating moiety, (1d) detecting photon emission from the light-generating moiety (e.g. over an area of the substrate), and (1e) determining the effectiveness of the plant treatment agent based on the amount of detected photon emission and/or the fraction of the substrate area from which photon emission is detected. Alternatively the methods can further involve (2c) after a period of time in which the disease can grow, detecting photon emission from an endogenous light-generating moiety, and (2d) determining the effectiveness of the plant treatment agent based on the detected photon emission. This invention further provides a process for producing a crop protection agent that is suitable for controlling a plant disease caused by a plant disease organism and comprises a plant treatment agent. This process comprises determining the effectiveness of the plant treatment agent in controlling the plant disease organism as indicated above. This invention also provides a method for controlling a plant disease caused by a plant disease organism (for example, a fungal plant pathogen). This method comprises determining that a plant treatment agent is effective for controlling the plant disease organism as indicated above; and applying to the plant or portion thereof to be protected, or to the plant seed or seedling to be protected, an effective amount of said plant treatment agent.

### BACKGROUND OF THE INVENTION

The control of plant diseases caused by plant pathogens is extremely important in achieving high crop efficiency in terms of yield or quality. Plant disease damage to ornamental, vegetable, field, cereal, and fruit crops can cause significant reduction in productivity and thereby result in increased costs to the consumer. Many products are commercially available for these purposes, but the need continues for new plant treatment agents, which are more effective, less costly, less toxic, environmentally safer or have different modes of action.

The identification of crop protection chemicals effective in the control of plant pathogens relies on the evaluation of disease and the control thereof, and is routinely based on the visual evaluation of the area of plant tissue infected. In some instances the compound is evaluated for either preventative and/or curative activity. Preventative activity is assessed when the test compound is applied prior to infection with the disease organism and curative activity is assessed when the test compound is applied following infection with the disease organism. For representative methods for assaying preventative control of plant diseases, see U.S. Pat. No. 5,747,497, Tests A through E. For representative methods for assaying curative control of plant diseases, see U.S. Pat. No. 3,954,992, Examples 3, 4 and 9. Visual assessment of disease relies on assigning a quantitative value, such as an estimated percentage of plant area covered by disease or a numeric class designation, to a qualitative assessment (e.g., mild or severe). Extensive training with the individual disease is necessary for accurate assessment and to ensure that observational bias does not influence this type of evaluation. Observational bias and subjective drift in scoring are likely to decrease accuracy of the evaluation as the number of samples increases with high-throughput chemical screening strategies.

Digital image analysis in plant pathology has been associated with determinations of early symptoms of crop diseases on a field level (Blazquez, C. H. 1990, Plant Dis. 74: 589-592; Everitt, J. H. et al., 1999, Plant Dis. 83: 502-505). Advances in computer-based imaging technology have resulted in the development and application of imaging analysis for disease evaluation on individual plants or leaves. Most applications of imaging analysis to the evaluation of plant disease have relied on the detection of inherent color differences in reflected light between the uninfected and infected regions of the plant in either the visible or infrared regions of the spectrum of electromagnetic radiation. Such methods are limited by the ability to distinguish between minute differences in color resulting from infection and require the development of extensive disease symptomology sufficient for detection and assessment. Recently, approaches relying on the non-invasive detection of light generated by bioluminescence or fluorescence have been used for the microscopic study of cell biology-level events that are part of the plant host-pathogen interactions (Dane, F. et al., 1994, Hort Science 29: 1037-1038; Spellig, T. et al., 1996, Mol Gen Genet 252: 503-509; Duncan, K. E. et al., 1997, Phytopathology 87: S26), and to determine fungal biomass in soils (Morris, S. J. et al., 1996, Applied Soil Ecology 6: 161-167). U.S. Pat. No. 5,650,135 discloses the use of non-invasive, macroscale imaging of light-emitting conjugates to detect mammalian pathogens within the animal.

Thus a means for the sensitive and reliable detection and measurement of disease on plants and the control thereof by plant treatment agents by an objective imaging methodology is needed. Furthermore, the ideal imaging methodology would rely not on color discrimination, but rather on the differential production of electromagnetic radiation and the subsequent detection of light emission. The methods of the present invention provide an objective, sensitive and non-invasive approach to detect, localize and measure the extent of plant disease for the purpose of evaluating the effectiveness of a plant treatment agent.

### SUMMARY OF THE INVENTION

Methods are provided for determining the effectiveness of a plant treatment agent in controlling a plant disease organism. As further disclosed, the methods involve detecting photon emission from light-generating moieties. The methods involve (a) applying the plant treatment agent to a substrate, (b) inoculating the substrate with the disease organism, and detecting photon emission from light-generating moieties. It is noted that (a) and (b) may be accomplished in either order. Generally, when (a) is accomplished before (b), the effectiveness of the plant treatment agent in preventing disease is assessed and when (b) is accomplished before (a), the effectiveness of the plant treatment agent in curing disease is assessed.

In one embodiment (Embodiment I) the method further includes (1c) after a period of time in which the disease organism can grow, applying a pro-light-generating moiety (a “PLGM”) which is selectively transformed by the disease organism or the substrate to a light-generating moiety (a “LGM”), (1d) detecting photon emission from the light-generating moiety (e.g. over an area of the substrate), and (1e) determining the effectiveness of the plant treatment agent based on the amount of detected photon emission and/or the fraction of the substrate area from which photon emission is detected. Of note are methods where the substrate is a plant substrate and a pro-light-generating moiety is applied after a period of time in which the disease can be manifested in the plant substrate.

In another embodiment (Embodiment II) the method further includes (2c) after a period of time in which the disease can grow, detecting photon emission from an endogenous light-generating moiety, and (2d) determining the effectiveness of the plant treatment agent based on the detected photon emission. This method is characterized by the substrate having an endogenous light-generating moiety not present in the disease organism and/or the disease organism having an endogenous light-generating moiety not present in the substrate. Of note are methods where the substrate is a plant substrate and the period of time is one in which the disease can be manifested in the plant substrate.

This invention further provides a process for producing a crop protection agent that is suitable for controlling a plant disease caused by a plant disease organism and comprises a plant treatment agent. This process comprises determining the effectiveness of the plant treatment agent in controlling the plant disease organism as indicated above.

This invention also provides a method for controlling a plant disease caused by a plant disease organism (for example, a fungal plant pathogen). This method comprises determining that a plant treatment agent is effective for controlling the plant disease organism as indicated above; and applying to the plant or portion thereof to be protected, or to the plant seed or seedling to be protected, an effective amount of said plant treatment agent.

### BRIEF DESCRIPTION OF THE FIGURES

FIG. 1 is a diagram of a macro imaging system for detecting light generating moieties.

FIG. 2 represents a map of a plasmid that can be used for the transformation of Magnaportha grisea to generate a disease organism expressing an endogenous LGM, the green fluorescent protein (GFP).

FIG. 3 represents a map of the plasmid pSLA2 that can be used for the transformation of tobacco to produce a plant substrate expressing an endogenous LGM, firefly luciferase.

FIG. 4 relates to Example 1 and represents the imaging detection of the effect of treatment with the commercial fungicide flusilazole on Erysiphae graminis (powdery mildew) infected on barley and made fluorescent by differential staining with the PLGM, fluorescein diacetate.

FIG. 5 also relates to Example 1 and represents the results of tests of the instrument and operating variability for the use of light emission to detect and measure the extent of plant disease.

FIG. 6 relates to Example 2 and represents the effect of treatment with commercial fungicides on the plant pathogen Magnaportha grisea grown vegetatively in microtitre plate wells and made fluorescent with the addition of fluorescein diacetate.

FIG. 7 relates to Example 5 and represents the effect of treatment with the commercial fungicide ridomil on Phytophthora infestans infected on tomato and measured by detecting fluorescence from the endogenous LGM chlorophyll.

FIG. 8 relates to Example 6 and represents the detection and distribution of the bioluminescence from excised uninfected 35S::LUC::NOS3′ tobacco leaves, and from leaves infected with Sclerotinia sclerotiorum.

FIG. 9 relates to Examples 8 and 9 and represents a map of the plasmid pSM619 that can be used for the transformation of Magnaportha grisea to generate a disease organism expressing an endogenous LGM, a reef coral green fluorescent protein (ZsGreen FP).

FIG. 10 relates to Example 8 and represents reference and fluorescence images representing the detection and distribution of the ZsGreen FP-expressing Magnaportha grisea pathogen strain MG619 infected on barley.

FIG. 11 relates to Example 9 and represents the effect of treatment with commercial fungicides on the plant pathogen Magnaportha grisea made fluorescent by expression of the ZsGreen Fluorescent Protein and grown vegetatively in microtitre plate wells.

### DETAILED DESCRIPTION OF THE INVENTION

This invention pertains to methods that can be used to detect the extent of growth of a plant disease organism on a substrate for the purpose of evaluating the effectiveness of a plant treatment agent. The methods involve detecting photon emission from light-generating moieties. Embodiments I and II of the invention both pertain to methods which involve the application of the plant treatment agent to a substrate and the inoculation of the substrate with a disease organism. After a period of time in which the disease organism can grow, either a pro-light-generating moiety that is selectively transformed by either the disease organism or the substrate to a light-generating moiety is applied and the resulting light is subsequently detected (Embodiment I), or light may be detected from an endogenous light-generating moiety present differentially in either the disease organism or the substrate (Embodiment II). The plant treatment agent may be a known agent (which may be evaluated for a number of purposes) or a substance not previously known as an effective control agent for the plant disease organism with which the substrate is inoculated (see Section I below). The substrate may be a plant (or a portion of a plant such as a leaf) or may be a non-plant medium adapted to support growth of the plant disease organism with which it is inoculated (see Section II below). While plant pathogens, in general, may be used in the methods of this invention, the methods are considered particularly suitable for determining the effectiveness of plant treatment agents in controlling fungal pathogens (see Section III below). Suitable PLGMs used in this invention are selectively transformed to LGMs, and include, for example compounds, such as fluorescein diacetate, that are transformed into the fluorescent fluorescein molecule. Of note are PLGMs that are selectively transformed by fungal pathogens to fluorescent LGMs (particularly when used with plant substrates). Also of note are PLGMs that are selectively transformed by plant substrates. Disease organisms or substrates having endogenous LGMs may also be used. Endogeneous LGMs may be naturally present (e.g., chlorophyll) or may be introduced (e.g. by genetic transformation) into a disease organism and/or living substrate (see Section IV below). Of note are fungal pathogens that have endogenous fluorescent LGMs. Also of note are plant substrates that have endogenous bioluminescent LGMs.

The light generated by the LGM (i.e., photon emission) is used in accordance with this invention to determine the effectiveness of the plant treatment agent. Photon detectors may be employed in a number of ways to practice this invention. A photon-detecting device is preferably used to detect the photon emission from the light-generating moiety and the effectiveness of the plant treatment agent may be determined based on the photon emission detected by such a device (See Section V). Various means of imaging and photon emission analysis may be employed to determine the effectiveness of the plant treatment agent (see Section VI). One means to determine the effectiveness of the plant treatment agent is to measure the amount of photon emission. For example, when a PLGM is selectively transformed by the disease organism to an LGM or when the disease organism has an endogenous LGM that is not present in the substrate, one may compare the total amount of photon emission from an inoculated substrate (e.g. a non-plant growth medium inoculated with a plant disease organism or an standardized plant substrate inoculated with a plant disease organism) treated with a plant treatment agent with the total amount of photon emission from (i) an equivalent inoculated substrate that is not treated with a plant treatment agent (i.e., an untreated control), (ii) an equivalent inoculated substrate that is treated with a known plant treatment agent of known effectiveness in controlling the disease organism (i.e., a standard) and/or (iii) an equivalent non-inoculated substrate (i.e., a blank). Preferably comparison with at least one untreated control, at least one standard and at least one blank is used. Analogously, when a plant substrate has an endogenous LGM not present in the plant disease organism, then the total amount of photon emission may be used to indicate the effectiveness of the plant treatment agent except that the light emitted is indicative of the portion of the plant that is not affected by the plant disease organism. Another means to determine the effectiveness of the plant treatment agent is to measure the fraction of the substrate area from which photon emission is detected. For example, when a PLGM is selectively transformed by the disease organism to an LGM or when the disease organism has an endogenous LGM that is not present in the substrate, one may compare the total area from which photons are emitted by said LGM to the total area of the inoculated substrate treated with a plant treatment agent to determine the fractional area affected by the disease organism. Preferably, this result is compared to the fractional area affected by the disease organism obtained from (i) an equivalent inoculated substrate that is not treated with a plant treatment agent (i.e., an untreated control), (ii) an equivalent inoculated substrate that is treated with a known plant treatment agent of known effectiveness in controlling the disease organism (i.e., a standard) and/or (iii) an equivalent non-inoculated substrate (i.e., a blank). Analogously, when a plant substrate has an endogenous LGM not present in the plant disease organism, then the fractional area of photon emission may be used to indicate the effectiveness of the plant treatment agent except that the light emitted is indicative of the portion of the plant that is not affected by the plant disease organism.

A plant treatment agent is identified as effective if it is able to significantly inhibit growth of the disease in experimental samples preferably relative to control samples and/or in comparison to samples treated with chemical standards of known efficacy. Growth of the disease organism is further defined, depending on the application, as the fractional area and/or distribution of the disease organism in or on the substrate, or the fractional area of damaged plant substrate resulting from the infection process versus undamaged plant substrate, or as the accumulated amount of the disease organism.

Accordingly, through use of light-generating moieties and photon-detecting devices the methods can be used to provide an objective means of evaluating the effectiveness of treatment agents in the control of plant disease. In addition, the use of photon-detecting devices can often enhance the detection of disease, thus providing a means for more sensitive and, in some cases, earlier determination of the effectiveness of a plant treatment agent.

In addition, the methods of this invention may be used in connection with an imaging approach that is non-invasive. This permits a user to track the extent and localization of the disease organism over time, by repeating the imaging steps at selected intervals, and constructing images corresponding to each of those intervals. This aspect of the methods is particularly useful for determining the effectiveness of a plant treatment agent in a curative application. Computer-based digital image analysis of diseased plant material can generate an objective quantitative evaluation. This invention nevertheless may be used for detecting the level of a disease organism without necessarily localizing the subject in the form of an image. This might be useful for the evaluation of the effectiveness of plant treatment agents, for example, when the area of the substrate is uniform.

The methods of this invention for detecting the effectiveness of plant treatment agents may be incorporated as an important step in the production of crop protection agents for controlling plant diseases containing effective plant treatment agents (see Section VII) and their use (see Section VIII).

I. Plant Treatment Agent

The plant treatment agent is a substance that is tested for agronomic utility as an active component of a crop protection agent effective in the control of plant disease (e.g., fungicides, antimicrobial and antiviral agents, and/or inducers of systemic acquired disease resistance), and may be a chemical compound or mixture of chemical compounds (e.g., a chemical mixture resulting from a physical mixing process or a mixed chemical synthesis process, a fermentation broth, or an extract preparation from a biological or non-biological origin). Compounds known to be fungicidal include acibenzolar S-methyl, azoxystrobin, benomyl, blasticidin-S, Bordeaux mixture (tribasic copper sulfate), bromuconazole, carpropamid, captafol, captan, carbendazim, chloroneb, chlorothalonil, copper oxychloride, copper salts, cymoxanil, cyproconazole, cyprodinil, (S)-3,5-dichloro-N-(3-chlor-1-ethyl-1-methyl-2-oxopropyl)-4-methylbenzamide (RH 7281), diclocymet (S-2900), diclomezine, dicloran, difenoconazole, fenamidone (RP 407213), dimethomorph, diniconazole, diniconazole-M, dodine, edifenphos, epoxiconazole, famoxadone, fenarimol, fenbuconazole, iprovalicarb, fenpiclonil, fenpropidin, fenpropimorph, fentin acetate, fentin hydroxide, fluazinam, fludioxonil, flumetover (RPA 403397), fluquinconazole, flusilazole, flutolanil, flutriafol, folpet, fosetyl-aluminum, furalaxyl, furametapyr (S-82658), hexaconazole, ipconazole, iprobenfos, iprodione, isoprothiolane, kasugamycin, kresoxim-methyl, mancozeb, maneb, mefenoxam, mepronil, metalaxyl, metconazole, metominostrobin, myclobutanil, neo-asozin (ferric methanearsonate), oxadixyl, penconazole, pencycuron, probenazole, prochloraz, propamocarb, propiconazole, pyrifenox, pyrclostrobin, pyrimethanil, pyroquilon, quinoxyfen, spiroxamine, sulfur, tebuconazole, tetraconazole, thiabendazole, thifluzamide, thiophanate-methyl, thiram, triadimefon, triadimenol, tricyclazole, triticonazole, validamycin and vinclozolin. The substance to be evaluated can also be an organism (e.g., virus, bacterium, fungus) potentially capable of attacking the target disease organism. The methods of this invention may be used, for example, to confirm the effectiveness of known plant treatment agents such as those disclosed above in known applications, or to evaluate their effectiveness for new applications. However, the methods of this invention are considered particularly useful for determining the effectiveness of agents not previously known as effective for fungal control. In the assay, the plant treatment agent being evaluated may be applied as a foliar treatment to the plant using conventional spray technology, drench or drip methods. One preferred foliar application method is by the use of a spray apparatus specially designed for the efficient application of microgram amounts of the plant treatment agent (see U.S. Patent Application No. 60/172,928, and its counterpart European Application Publication No. EP1110617 which are hereby incorporated by reference herein). Alternatively, the plant treatment agent may be applied as a systemic treatment directly to the growth medium, followed by the subsequent uptake by the plant and presentation to the disease organism. Known plant treatment agents may be used as controls in connection with the evaluation of new agents to provide a relative measure of effectiveness.

II. Substrate

The substrate may be any material suitable for growth of the plant disease organism. Exemplary substrates include plant substrates (i.e., plants including monocots and dicots, such as grasses and broadleaf plants, woody plants and trees, agronomic crop species, and weed species and parts of such plants). Of note are standardized plant substrates, i.e., plant substrates that are uniform with respect to such factors as species, size, age and/or stage of development so that the different responses to different treatments can be attributed to the differences in treatment. Preferred methods of this invention that use plant substrates include those using cultivars of Arabidopsis thaliana, barley, cucumber, grape, lovegrass, maize, potato, rice, squash, tomato, or wheat. The substrate may also be a liquid, soil or other solid growth medium sufficient to support the metabolism of the disease organism. Typically the substrate is held in a container or on a support. Examples include containers used for the horticultural production of plants, test tubes, jars, wells of microtitre plates, or flat supports such as glass or plastic plates. The containers may also contain or support rock wool, or other inert support material.

III. Disease Organism

The disease organism is defined as a plant pathogen. Plant pathogens may include fungal, bacterial or viral agents that cause disease on plants. Preferred methods of this invention include those where disease organisms are fungal pathogens. Example fungal pathogens include Alternaria, Fusarium, Monilinia, Plasmopora, Pseudocercosporella, Puccinia, Pyrenophora, Rhyncosporum, and Sclerotinia species, and strains of Erysiphae cichoracearum, Erysiphae graminis, Magnaportha grisea, Phytophthora infestans, Pyricularia oryza, Septoria triticii, and Stagnospora nodorum.

IV. Pro- and Light-Generating Moieties

A pro-light-generating moiety (PLGM) is an entity that is metabolized or otherwise selectively transformed by the action of either the plant substrate or the disease organism to produce a light-generating moiety (LGM). For example, where a PLGM is selectively transformed by the disease organism, the light generated will indicate the presence of the disease organism. Light-generating moieties are typically molecules or macromolecules that emit light in the ultraviolet (UV), visible and/or infrared (IR) portion of the spectrum. Light is defined herein as electromagnetic radiation having a wavelength between about 300 nm and about 1100 nm.

The PLGMs and LGMs useful in the practice of the present invention may take a variety of forms, depending on the application and the specific disease/substrate combination. LGMs may generate light as a result of radiation absorption (e.g., fluorescent or phosphorescent molecules), or as a result of a chemical reaction (e.g., proteins that produce bioluminescence as a result of catalysis, and chemiluminescence).

Fluorescence-Based PLGMs and LGMs

Fluorescence is the luminescence emitted from a substance in a single electronically excited state, induced by excitation of the substance with light of a wavelength suitable to induce electronic transitions. The wavelength of the emitted light is longer than that of the exciting illumination (Stokes shift), because the excited electron relaxes to the lowest excited state, generating heat as a by-product, before emitting a fluorescent quantum and returning to the ground state.

The use of fluorescent molecules in the present invention is complicated by the requirement for light input to generate the luminescence. The light used to excite a fluorescent target may result in the fluorescence of substances other than the intended target. This is particularly true when the sample being imaged is as complex as the chemical milieu of a biological organism. Specificity of the fluorescence emission is achieved by the use of optical filters to restrict the spectral range of the excitation light striking the sample. An appropriately selected excitation filter blocks the majority of photons having a wavelength similar to that emitted by the fluorescent LGM and of other ancillary LGMs that may be present in the sample. A laser producing high intensity light near the excitation wavelength, but not near the emission wavelength can also be used to specifically excite the LGM, as in confocal imaging applications. Further, excitation of the sample may be achieved in confocal imaging by laser excitation with two photons of light of a longer wavelength, and therefore each photon has less energy. Known as two-photon excitation, this method reduces the amount of photobleaching, which often accompanies high-energy laser excitation of the fluorophore.

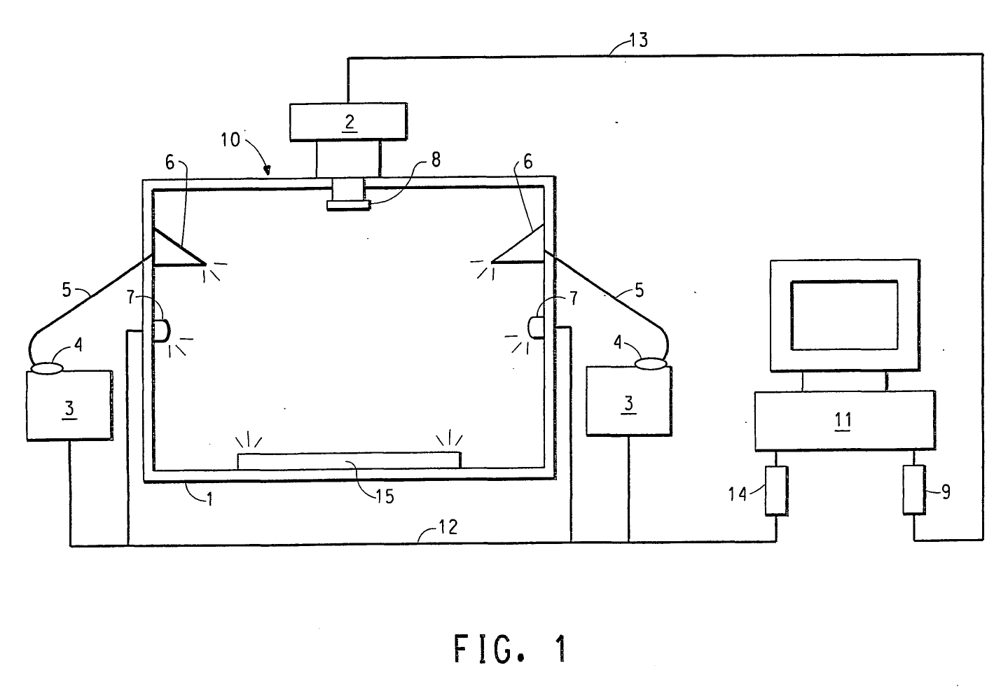

In addition, the spectral sensitivity of the photon-detecting device may be regulated by the addition of optical filters in front of the detector window to restrict the spectral range of light reaching the detector to those photons matching the emission wavelengths of that of the fluorescent LGM. Detectors may be selected that have reduced sensitivity to wavelengths of light used to excite the LGM. As an additional precaution, an imaging chamber suitable for housing the sample, excitation light and photodetector (e.g. a camera) may be used to prevent additional radiation sources (e.g., room light) from irradiating the sample and/or the photodetector during the integration period (see FIG. 1 for a macro imaging system comprising these elements).

Exemplary fluorescent light-generating moieties are small fluorescent molecules, such as fluorescein, used either in un-conjugated form and/or conjugated to antibodies or other proteins, polymers or carbohydrates; and fluorescent proteins (FP's), such as those from marine coelenterates (e.g. a green fluorescent protein). Of particular note for the present invention are the PLGM fluorescein diacetate (FDA), and the LGM ZsGreen FP.

The diacetate derivative of fluorescein is a useful compound for the study of live cells. FDA itself is non-fluorescent, but upon uptake into a live cell it is transformed into the LGM fluorescein. Fluorescein absorbs light maximally at about 475 nm and emits in the green region of the spectrum, with a maximum emission at about 517 nm.

FP's, unless stated otherwise, are defined as the fluorescent proteins from coelenterates. FP's include GFP, the green fluorescent protein from the marine jellyfish Aequoria victoria and includes engineered variants with altered optical properties such as color-shifted fluorescence and increased extinction coefficient for enhanced quantum yield of fluorescence, engineered variants with altered protein properties such as altered solubility in solution. FP's also include fluorescent protein variants isolated from other natural sources, such as AmCyan, ZsGreen, ZsYellow, DsRed, and AsRed Fluorescent Proteins (FP) isolated from related marine coelenterates (e.g. Zoanthus sp. or Discosoma striata) and their engineered sub-variants. The FP's, as defined here, are distinct from other naturally occurring fluorescent proteins, as the FP fluorescence is due entirely to an internal interaction between amino acids within the protein. The FP's require no other prosthetic groups or cofactors as are required for the fluorescent chromoproteins proteins, such as the chlorophyll-binding proteins.

Aequoria victoria are brightly luminescent, with light appearing as glowing points around the margin of the jellyfish umbrella. Light arises from yellow tissue masses which each consist of about 6,000 to 7,000 photogenic cells. The cytoplasm of these cells is densely packed with fine granules of about 0.2 μm diameter, which are enclosed by a unit membrane, and contain the components necessary for bioluminescence. The components include a Ca²⁺ activated photoprotein, aequorin, that emits blue-green light, and an accessory green fluorescent protein (GFP) which accepts energy from aequorin and re-emits it as green light. The GFP protein is intensely fluorescent with a quantum efficiency of approximately 80%. GFP absorbs light maximally at about 395 nm and has a smaller absorption peak at 470 nm, and fluorescence emission peaks at about 509 nm with a shoulder at about 540 nm. These optical properties make it suitable for use with argon laser-excited confocal microscopes and with epi-fluorescence microscopes equipped with common fluorescein filter sets. The protein fluorescence is due to a unique covalently attached chromophore, which is formed post-translationally by the cyclisation of the residues Ser-dehydroTyr-Gly within the protein. The gene encoding the green fluorescent protein has been cloned (Chalfie, M., et al., 1994, Science 263: 802-805) and successfully expressed in a wide range of heterologous organisms, including Escherichia coli, Caenorabditis elegans, Drosophila melanogaster, Saccharomyces ceriviae, mammals and plants (Chalfie, M., et al., 1994, Science 263: 802-805; Wang and Hazelrigg, 1994, Nature 369: 400-403; Haseloff and Amos, 1995, Trends Genet 11: 328-329; Cubitt et al., 1995, Trends Biochem Sci 20: 448-455; Baulcombe et al., 1995, Plant J 7: 1045-1053; Sheen et al., 1995, Plant J 8: 777-784). The genes for the FP's AmCyan, ZsGreen, ZsYellow, and AsRed have been cloned from non-bioluminescent reef corals using degenerate primers with homology to the A. Victoria GFP (Matz et al., 1999, Nature Biotechnology 17: 969-973). These FP's contribute the bright fluorescent color of many Anthozoa species, and have been proposed to provide protection from strong solar radiation, and in deep water dwelling species to aid in conversion of the predominant blue light to longer wavelengths more suitable for photosynthesis by algal endosymbionts.

Bioluminescence-Based LGMs

Bioluminescent molecules are distinguished from fluorescent molecules in that they do not require the input of radiation to produce light. Rather, bioluminescent molecules utilize chemical energy, such as ATP, to produce light. An advantage of bioluminescent LGMs, as compared to fluorescent LGMs, is that there is virtually no background light signal in the disease organism or substrate in the absence of an excitation light. The only light signal detected is that produced by the bioluminescent LGM.

A bioluminescent protein of note is luciferase. Luciferase as defined here includes prokaryotic and eukaryotic luciferases, as well as variants possessing varied or altered optical properties, such as luciferases with color-shifted luminescence. Prokaryotic luciferase and other enzymes involved in prokaryotic luminescence (lux) systems and their corresponding genes have been cloned from marine bacteria in the Vibrio and Photobacterium genera and from the terrestrial microorganism Photorhabdus luminescens. Expression of the luxCDABE operon from Photorhabdus luminescens provides the genes (luxAB) for synthesis of the bacterial luciferase enzyme which is optimally active at 37° C. and thermostable up to 47° C., and the genes (luxCDE) for the synthesis of the aldehyde substrate of the prokaryotic luciferase. Oxygen is the only extrinsic requirement for bioluminescence using the prokaryotic lux system.

An exemplary eukaryotic organism containing a luciferase system (luc) is the North American Firefly Photinus pyralis. Firefly luciferase has been extensively studied and has long been used in ATP assays. The cDNA for the luciferase gene (luc) has been cloned from the firefly, Photinus pyralis, (DeWet et al., 1985, PNAS 82: 7870-7873), and more recently has been expressed in a wide range of biological systems, including bacterial, plant and animal cells (DeWet et al., 1987: Mol Cell Biol 7: 725-737; DeWet et al., 1985, PNAS 82: 7870-7873; Ow et al., 1986, Science 234: 856-859). The light from firefly luciferase ranges in color from green to yellow (550 to 580 nm). The range of bioluminescent light emitted from the related luminous click beetle (Pyroporus plagiophthalamus), is larger than that emitted from fireflies, ranging from blue-green to orange (530 to 590 nm). The cDNAs of the genes responsible for the bioluminescent light of the luminous click beetle have also been cloned. The availability of luciferase systems with a range of spectral emissions allows for the selection of color variants optimized for the custom application of the present invention, as described below. The firefly luc system requires the expression of only a single gene for light production, which is a potential advantage for expression in eukaryotic systems. Another advantage of the firefly luciferase system as a reporter in some applications of the present invention derives from the optimal catalytic activity of the luciferase enzyme at the ambient room temperatures (e.g., at about 23 to 28° C.) used for cultivation of the disease organisms and plant substrates. The firefly luciferase system requires the presence of the substrates O₂, ATP and luciferin for catalysis. In the typical application of the luc system, O₂ and ATP are derived from the biological tissue and firefly luciferin ((S)-4,5-dihydro-2-(6-hydroxy-2-benzothiazolyl)-4-thiazolecarboxylic acid) is supplied to the biological sample, depending on the organism and test format, either by spraying, feeding, watering, injection, or other means.

LGM Spectral Properties

The preferred PLGMs and LGMs of this invention have spectral properties that facilitate photon detection (i.e., they produce light of a wavelength that can be distinguished from light generated by the disease organism and/or substrate, depending on the application). The spectral properties of the LGMs selected for use in the disease organism are particularly important when the substrate is a plant. Plants naturally produce a wide range of LGMs with varying spectral properties and which accumulate to varying extent dependent upon plant species, growth conditions and disease status. Plant LGMs include chromoproteins and pigments, such as chlorophyll, carotenoids, and anthocyanins, some aromatic amino acids, and some plant secondary metabolites. By far the most abundant of the plant LGMs are the chlorophyll-containing pigment-protein complexes of the plant photosynthetic machinery. Chlorophylls are fluorescent molecules which absorb light at two wavelength maxima, one in the blue end of the visible spectrum, at about 430 nm, and a broader absorption band in the red wavelengths, with a maximum around 660 nm. The resulting chlorophyll fluorescence emission is in the red and far-red range of the visible spectrum. Chlorophyll absorbs little light energy in the green wavelengths, and notably plants reflect most green light back to the environment, resulting in the typical green color for most plant species. Hence, for some applications of the present invention a LGM must be selected which emits and/or absorbs light at a wavelength that does not overlap significantly with the absorption and emission wavelengths for the endogenous LGM's and especially the abundant chlorophyll pigment-protein complexes.

In applications of the present invention with a plant substrate and employing fluorescent LGMs localized in the disease organism, the LGM should optimally absorb in the blue to green wavelengths and emit in the green region of the spectrum so as to avoid spectral overlap with the predominant chlorophyll pigments. As described above, the absorption and emission spectra for the PLGM-derived fluorescein and for GFP, including the engineered GFP variants that have been modified to absorb maximally at a range of about 473 to 489 nm and fluoresce at approximately 510 nm, and for FP's such as ZsGreen fall into the spectral gap for chlorophyll absorbance. Fluorescein, GFP and ZsGreen FP produce light at wavelengths predominantly reflected by the plant rather than absorbed, and are therefore particularly well suited for use in the present invention.

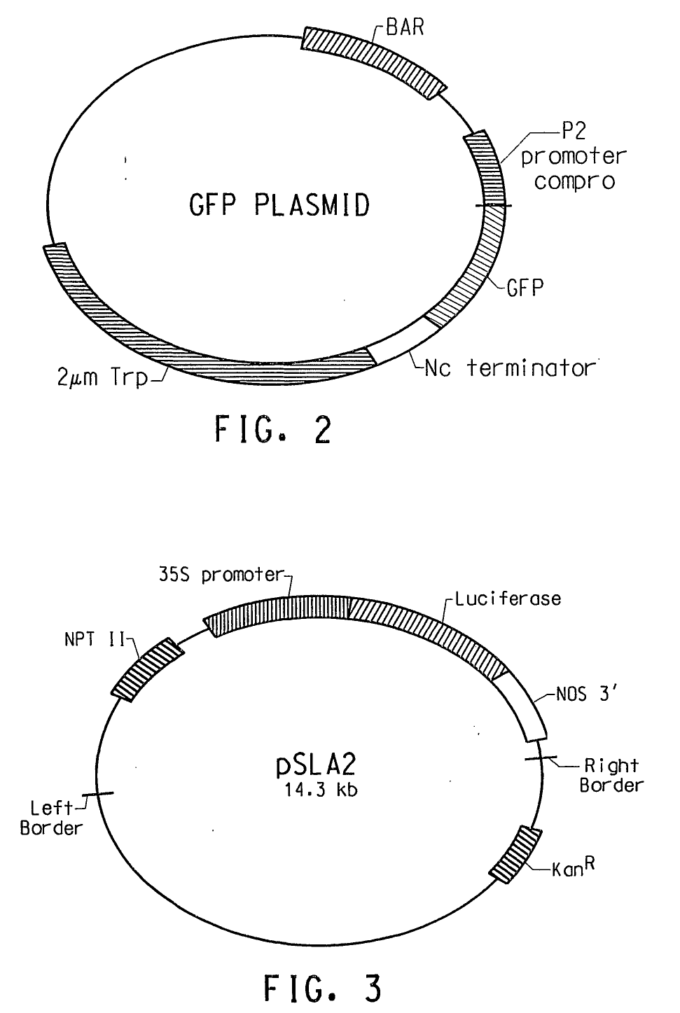

Construction of a GFP-expressing Magnaportha grisea strain can be carried out as described in the following. A series of plasmids referred to as “compro” (because all the plasmids in the series have a common 20 bp sequence at the 3′ end of the promoter) can be constructed according to standard molecular biology techniques well-known to those skilled in the art. For example, such plasmids are based on pBluescript and containing a fungal promoter with the “compro” sequence, the Neurospora crassa B-tubulin transcription terminator, the bialaphos resistance gene for fungal selection, trp1 of Saccharomyces cerevisiae, and a 2 μm yeast origin of replication. A version containing the M. grisea P2 ribosomal protein promoter is pSM324. Plasmid pSM324 can be digested with Xho I and the 5′ overhang filled-in with the Klenow fragment of DNA polymerase I. Of use in the present invention are GFP variants engineered for enhanced fluorescent yield relative to wildtype GFP, such as EGFP (available from Clontech Laboratories, Inc. Palo Alto, Calif.) and rsGFP (available from Quantum Biotechnologics, Inc. Montreal, Quebec, Canada). To generate a GFP expression vector, polymerase chain reaction is used to amplify the GFP coding sequences with 5′ and 3′ primers designed to the respective ends of the GFP sequence and with sequence extensions homologous to the pSM324 sequence flanking the Xho I site and sufficient for homologous recombination. For example, the primers “XFP5” (aggaacccaatcttcaaaatggtgagcaagggcgag) and “XFP3” (aatgttgagtggaatgatttacttgtacagctcgtcc), designed to the Clontech EGFP sequence, can be used for construction of a vector for expression of GFP in M. grisea. The XFP5/XFP3 amplification product and the Xho-digested pSM324 can then be transformed into Saccharomyces cerevisiase strain W303-1A by the lithium acetate method (Agatep et al., 1998) to allow gap repair of the plasmid. Yeast colonies can be selected on tryptophan-minus plates. Plasmid minipreps can be performed on yeast tryptophan prototrophs and an aliquot transformed into E. coli strain DH10B by electroporation. Plasmid DNA can be prepared from ampicillin-resistant E. coli transformants and screened by restriction digest for the proper gap repair of the XFP5/XFP3 amplification product into pSM324 to generate the GFP expression vector (FIG. 2). The GFP expression vector can be transformed into M. grisea strain 4091-5-8 using published transformation protocols (Sweigard et al., 1992). Bialaphos-resistant transformants of M grisea can be selected and purified by single spore selection. Independent transformants can be screened for fluorescent intensity and an exceptionally bright transformant can be selected for further use.

In addition, plant secondary metabolites whose accumulation is induced upon infection by plant pathogens and which are fluorescent are of relevance to the present invention. Such compounds include lignin, sinapoyl malate and other phenylpropanoid derivatives, among others. Thus, it may be desirable to select a LGM that does not overlap with the absorption and emission wavelengths of such secondary metabolites. In certain cases where the light level from fluorescent secondary metabolites is low in comparison to the light levels produced by the LGM of the invention, the luminescence derived from the secondary metabolites may be discounted and/or corrected for by comparison to the signal from the appropriate controls (e.g. diseased host plants lacking the LGM detected in the method).

Similar considerations of spectral properties apply to the use of bioluminescent LGMs. Observation of bioluminescence may be hindered by absorbance of the bioluminescent light by the plant substrate or the disease organism. Plant tissue, as described above, does not quantitatively absorb green light. Hence, bioluminescence in the green region of the spectrum, as produced by the luciferase/luciferin reaction, will not be significantly quenched by chlorophyll absorption, but rather will be optimally transmitted through the plant tissue. Optimal transmission of light, whether arising from fluorescence or bioluminescence, through the plant substrate is particularly important in the application of the present invention for the assay of disease organisms which grow internal to the plant leaf, rather than primarily exposed on the leaf surface.

Differential PLGM and LGM Accumulation

Methods for the differential localization and accumulation of PLGMs or LGMs between the disease organism and substrate can be critical for the successful application of the present invention. In certain embodiments it is the differential accumulation of and the subsequent detection of the LGM that forms the basis of the method for detecting the extent of disease organism growth.

The PLGM or LGM can be differentially conjugated to the disease organism or substrate by a variety of techniques, which include differential application to or accumulation in the disease organism and/or substrate by injection, pipeting, watering, submersion or spraying of a small molecule or chemical. Such methods may require subsequent redistribution and/or exclusion of the PLGM or LGM by the action of the biological organisms (e.g., Fluorescein diacetate, Embodiment I). The LGM may be an endogenous component of either the disease organism and/or plant substrate, which differentially accumulates. Endogenous is defined herein as a property or entity originating or developing within either the disease organism or the plant substrate, and includes proteins expressed as the result of genetic transformation of the organism with a heterologous gene. One example is the biosynthesis and disruption thereof of an endogenous PLGM or LGM during growth of the disease organism and/or plant substrate (e.g. chlorophyll biosynthesis, Embodiment II). Alternatively, the disease organism and/or plant substrate may be engineered for in situ synthesis of the LGM (e.g., expression of a heterologous fluorescent protein in a transformed cell, regulated by either a constitutive promoter or an inducible promoter controlled by the administration of the appropriate promoter inducer, Embodiment II).

Applied Small Molecule Fluorescent PLGMs and LGMs

It is often desirable to identify a small molecule PLGM or LGM that solely on the basis of biophysical and/or chemical properties can localize and specifically accumulate to a significant extent in either the disease organism or the substrate. The PLGM fluorescein diacetate has chemical properties that enable it to preferentially accumulate in and be transformed to a LGM in some fungal disease organisms relative to the plant substrate. The lipophilic diacetate moiety allows the FDA molecule to permeate cell membranes. Once inside the cell, the diacetate groups are cleaved by non-specific cellular esterases, forming a charged form of the fluorescent fluorescein molecule that leaks out of the cell far more slowly than the parent compound.

Many fungal pathogens of plants possess surfaces, cell walls and membranes through which FDA is readily permeant, and they contain sufficient cellular esterase activity for the quantitative conversion of FDA to fluorescein. In comparison, the external surfaces of plant leaves are covered by layers of epicuticular wax and are characteristically hydrophobic. For fungal pathogens of plants that grow primarily exposed on the leaf surface, such as powdery mildew (Erisyphae graminis), FDA applied to the surface is readily accessible to the pathogen, but it can not penetrate the plant substrate. Other pathogens, such as Puccinia graminis, the causative agent of leaf rust, produce disease lesions with fruiting structures exposed on the leaf surface, but do not become highly fluorescent upon FDA application, likely due to pathogen-specific differences in FDA uptake and/or metabolism. FDA is excluded from the leaf by the hydrophobic epicuticular waxes and as a result is inaccessible to pathogens that grow primarily internal to the leaf. Some pathogens may be induced to sporulate under appropriate environmental conditions (i.e., high humidity), producing fruiting structures on the leaf exterior that are accessible to FDA staining. Magnaportha grisea, the causative agent of rice blast disease, is an exemplary pathogen that grows internal to the leaf blade but can be differentially labeled with FDA following sporulation.

Thus, the combination of chemical properties of the FDA/fluorescein pair, the biophysical differences in hydrophobicity of the cell membrane of the disease organism relative to the substrate, and the growth habit of the pathogen relative to the leaf surface regulates the differential accumulation of fluorescein in the disease organism. An individual skilled in the art of the present invention is able to identify disease organism/substrate pairs with the appropriate combination of growth habits and biophysical traits (e.g., surface pathogen capable of FDA uptake) for which FDA is the preferred PLGM.

For the present invention, FDA is typically formulated in a dilute detergent working solution that aids in dispersing the FDA in solution and on the hydrophobic leaf surface. In the assay, the FDA may be applied as a foliar treatment to the plant using conventional spray technology, drench or drip methods. The FDA working solution is preferably sprayed as a fine mist onto intact plants to avoid displacing fungal fine structure and is applied until run-off is observed. Commercially available Preval® sprayers (Precision Valve Corporation, Yonkers, N.Y.) have proven effective for this application. In addition, airbrush paint applicators also provide a suitably fine mist for this application.

Endogenous LGMs

Upon infection with the disease organism many host plants undergo the process of localized cell death in an attempt to curtail the spread of the infection. In some cases, the disease disrupts the host's normal cellular processes and/or causes cell death. As a result of the infection and disease progression process, the biosynthesis and accumulation of an endogenous LGM such as chlorophyll may be altered. The differential chlorosis resulting from this pathology can be used as a marker for disease growth. Hence, in another application of the invention, the predominant fluorescence signal from the chlorophyll pigment is used advantageously to detect the uninfected, healthy regions of the plant substrate. By subtracting the area of chlorophyll-fluorescent, healthy leaf tissue from the total area of the leaf determined from a reference image, the area infected by the disease organism may be determined.

Another method for the differential accumulation of an endogenous PLGM or LGM is by the expression of a heterologous gene encoding a light-generating protein in either the disease organism or the plant substrate. Heterologous genes are genes that have been transformed into a host organism. Typically, a heterologous gene is a gene not originally derived from the transformed host organism's genomic DNA. In the application of the present invention the preferred heterologous genes are those encoding the LGMs luciferase and FP's with emission in the green wavelengths, such as GFP or ZsGreen FP, as described above. This method is applicable to disease organisms and host plant species in which genetic transformation is possible. This method includes administering to a subject (either disease organism or plant host) a vector construct effective to integrate a transgene into the subject's cells. Such vector constructs are well known to those skilled in the art. Examples of vector constructs are represented by the plasmid maps of FIGS. 2, 3 and 9. In addition to elements necessary to integrate effectively, the construct contains a transgene (e.g. a therapeutic gene or selectable marker) and a gene encoding the LGM under the control of a selected activatable promoter.

With the expression of the luciferase or FP (e.g. GFP or Zs Green FP) reporter genes in the disease organism, the organism becomes bioluminescent or fluorescent, respectively, and the resulting light generation can be used to directly detect the extent of infection by the disease organism. A constitutive promoter may be used to drive expression throughout the disease organism permitting detection of the organism whether it grows on the leaf surface or internal to the leaf blade. Inducible promoters may be used to drive expression only when desired for disease detection and in response to a promoter induction event. Promoter induction events include the administration of a substance that directly activates the promoter, the administration of a substance that stimulates the production of an endogenous promoter activator, the imposition of conditions resulting in the production of an endogenous promoter activator (e.g. heat shock or stress), and the like. In addition, tissue-specific promoters directing the expression of the LGM solely in the reproductive structures of the pathogen may be used to detect the ability of a plant treatment agent to prevent the spread of disease by inhibition of the infection cycle.

Alternatively, the expression of luciferase or FP (e.g. GFP or Zs Green FP) in the host plant renders the host tissue light generating. As with the use of the endogenous LGM chlorophyll described above, the host plant expression of a heterologous gene encoding a LGM can be used to detect healthy, uninfected regions of the host plant. As a result of the infection and disease progression process, the expression and accumulation of a heterologous LGMs such as luciferase or FP may be altered. The differential reporter gene expression and light generation resulting from this pathology may be used as a marker for disease growth. Similar to the method of luciferase or FP expression in the disease organism, inducible promoter systems may be used to direct the reporter gene expression in the host plant, and may include host promoters induced in response to infection with a pathogen.

V. Photon Detectors

A photon-detecting device is an instrument that can detect photon emission. Photon-detecting devices may include the unaided human eye, the human eye aided by the use of night vision goggles, a fluorescence scanner, a fluorometer or spectrophotometer, a digital camera, a photomultiplier (PMT), a charge coupled device (CCD), or a time-delay integrating (TDI) CCD that detects photons from an object as it travels relative to the detector. An important aspect for many applications of the present invention is the selection of a photon-detecting device with sufficient sensitivity to enable the detection and imaging of faint light from within or on a plant in a reasonable amount of time to provide sufficient sample throughput. For example, after a period of time in which the PLGM or LGM can differentially localize in the subject and in which the PLGM is transformed by the subject into a LGM, the subject may be held within the detection field of a photon-detecting device for a length of time necessary to measure a sufficient amount of photon emission to construct an image. In cases where it is possible to use LGMs that are extremely bright, a pair of night-vision goggles or a standard high-sensitivity video camera, such as a Silicon Intensified Tube (SIT) camera may be used. More typically, however, a more sensitive method of light detection is required.

With extremely low light levels, such as those typically encountered in the practice of the present invention, the photon flux per unit area can become so low that the scene being imaged no longer appears continuous. Instead, it may be represented by individual photons, which are both temporally and spatially distinct from each other. Viewed on a monitor, such an image typically appears as scintillating points of light, each representing a single detected photon. Nevertheless, by accumulating these photons in a digital image processor over time, an image can be acquired.

At least two types of photon-detecting devices, described below, can detect individual photons and generate a signal that can be analyzed by an image processor.

Photon Amplification Devices

This class of sensitive photon-detecting devices employs additional devices to intensify single photon events before they reach the detector. This class includes CCD cameras with intensifiers, such as microchannel intensifiers. An exemplary microchannel intensifier-based single photon detection device is the C2400 system, available from Hamamatsu Photonic Systems (Bridgewater, N.J.). A microchannel intensifier typically contains a metal array of channels perpendicular to, or at a slight angle to, the detection screen, which is co-extensive to and positioned in front of the detector screen. A photocathode device is positioned between the microchannel array and the sample. A photon striking the photocathode causes the ejection of an electron, which enters the microchannel array. Most of the electrons that enter the microchannel array contact a side of the channel before exiting. A voltage applied across the array results in the release of many electrons for each electron collision. The resulting electron clouds exit the microchannel array and are detected by the camera. Even greater sensitivity can be achieved by placing intensifying microchannel arrays in series, resulting in an even greater amplification of the original photonic signal. However, it is noted that the increase in sensitivity achieved by the use of microchannel arrays to intensify the photonic signal is gained at the expense of spatial resolution. Some applications of the present invention that detect area of coverage by a disease organism as the metric for growth require high spatial resolution.

Reduced-Noise Photon-Detection Devices

This class of photon-detecting devices achieves sensitivity by reducing the background noise in the detector, as opposed to amplifying the signal. Noise is primarily reduced by cooling the detector array, thereby reducing the dark current (i.e., electrical current that results from leaks in the circuitry of the instrument) and, most significantly, accumulates at the detector head. The deeper the cooling (by, for example, liquid nitrogen, which can reduce the temperature of the CCD array to about −120° C.), the more sensitive the detector. More sensitive versions of these cooled devices include CCD arrays referred to as “backthinned” that may be operated in a back illuminated mode. “Backthinned” refers to an ultrathin backplate of the CCD array. Thinning the CCD array reduces the path length a photon must travel to be detected, and coupled with the back illumination, avoids light absorption by the polysilicon gates at the front of the CCD array, thus greatly improves the quantum efficiency of the detector. In addition, a new CCD technology called multi-pin phasing (MPP), by reducing the potential at the surface of the CCD during the exposure time, can reduce dark current by a factor of 100 or more. Detectors are available which employ all of these technologies (i.e., cooling, backthinned, back illuminated arrays, and MPP) for optimal camera performance. An exemplary reduced-noise photon-detection camera employing all of these technologies, and yet providing excellent high-resolution characteristics (1317×1035 imaging array with 6.8×6.8 μm pixels), is the SenSys® 1401E camera system, available from Roper Scientific (Tucson, Ariz.).

Camera systems are also available which combine both photon amplification and noise reduction technologies (e.g., a cooled, intensified CCD). Such camera systems are generally very sensitive for light detection, but provide somewhat lower spatial resolution than cameras employing solely noise reduction technologies.

Image Processors

Signals generated by photon-detecting devices that count single photons typically need to be processed by an image processor in order to construct an image that can be, for example, displayed on a monitor or printed. The detection of photon emission generates an array of numbers, representing the number of photons detected at each pixel location, in the image processor. These numbers are used to generate an image, typically by normalizing the photon counts (either to a fixed preselected value, or to the maximum number detected in any pixel in the field) and converting the normalized number to a brightness (grayscale) or to a color (pseudocolor) that is displayed on a monitor. In a grayscale presentation, typical color assignments are as follows. Pixels with zero photon counts are assigned black, low counts are assigned shades of gray, and white is assigned for pixels having the highest photon counts. The locations of the gray and white pixels on the monitor represent the distribution of photon emission, and, accordingly, the location of the light generating moieties.

Image processors are typically sold as part of systems that include the sensitive photon-counting cameras described above, and accordingly are available from the same sources (e.g., Hamamatsu and Roper Scientific). The image processor is usually connected to a personal computer, such as an IBM-compatible PC or an Apple Macintosh, that may or may not be included as part of the purchased camera system. After the images are in the form of digital files, they can be manipulated and analyzed with a variety of image processing programs, including software applications available from the camera system vendors, other commercial applications such as MetaMorph® (Universal Imaging, West Chester, Pa.) or Adobe Photoshop® (Adobe Systems, Mountain View, Calif.), or custom software applications.

Imaging System Integration

One macro imaging system that can be used in the present invention (see FIG. 1) is an integrated, computer-controlled instrument. In this example of the imaging system, the configuration is such that the samples are placed in the base (1) of the imaging chamber (10), and the camera (2) focuses downward. However, in some applications of the methods it may be desirable to have the camera mounted on the side of the chamber. Still other configurations of the camera relative to the samples to be imaged may be used for other applications of the methods.

When used in the fluorescence mode, the excitation light in this example is produced by dual light sources (3) (e.g. DCR® II LITE SOURCE available from Optical Apparatus Co., Inc., Ardmore, Pa.), each fitted with the appropriate bandpass filters (4) (for example filters available from Omega Optical, Brattleboro, Vt.) and coupled with a fiber optic bundle (5) to a 14″×0.015″ (35.6 cm×0.038 cm) fiber optic line light (6) (e.g. Lightline® available from Optical Apparatus Co., Inc., Ardmore, Pa.). The line lights (6) are mounted inside the imaging chamber out of the field of view of the camera, and oriented to provide flat-field illumination, without spectral reflectance, across the imaging field of view. Low wattage white lights covered by a diffuser (7) are also mounted inside the chamber and are used to provide flat-field, nonreflecting illumination for the collection of the reference images. Establishing uniform excitation and reference light conditions without spectral reflectance is important for some applications of the present invention, particularly when flat transparent materials such as Lucite® polycarbonate or Mylar® polyester are used to hold plant samples in a single plane for improved image accuracy, or when the host substrate contains reflective material, such as with a microtitre plate. Optionally, an additional flat-bed reference light (15) may be placed in the base of the imaging chamber (1) to backlight samples relative to the camera (2).

Emitted fluorescence, bioluminescence or reflected light may be detected by a camera (2) (e.g. a SenSys® 1401E cooled CCD camera (Roper Scientific, Tucson, Ariz.), fitted with a macro photographic lens, such as an AF Nikkor 35 mm f2.0 lens (available from Nikon, Inc., Melville, N.Y.), and an appropriate emission filter (8) (for example a filter available from Omega Optical, Brattleboro, Vt.), and connected to an image processor (9) having a 16-bit frame grabber.

Operation of the lighting systems for fluorescence and for reference images and of the CCD camera is controlled by a computer (11) through wire connections (12) and (13), respectively. A multifunction I/O board (14) (e.g. one available from National Instruments, Austin, Tex.) is installed in the computer for the control of the excitation and reference lights. Software drivers for the operation and control of the camera are generally provided by the camera manufacturer and are installed on the computer. In this example, a customized image analysis software program installed on a PC running the Microsoft Windows NT® operating system controls the lighting and camera operation.

VI. Image Acquisition and Analysis

Samples to be imaged are placed within the imaging chamber (10) (see FIG. 1), with orientation relative to the camera and dependent upon the substrate format (e.g., microtitre plate or plant leaf), and/or, when a plant is the substrate, upon the growth habit of the host plant (e.g., broadleaf or grass leaves). The field of view with a 35 mm macro lens may accommodate one or more plants, depending on their size, and one or more microtitre plates, depending on the working distance. Larger fields of view, and therefore increased throughput can be achieved with the use of shorter focal length lenses, such as 28 or 24 mm lenses. A field lens can be helpful for correcting parallax, especially when deep welled microtitre plates are used. However, such lenses generally are optically slow and may reduce the amount of light detected. In some applications of the methods, particularly when the substrate is a host plant with leaves that curl and otherwise present a non-uniform 3-dimensional target to be imaged, it may be desirable to flatten the sample to a single plane, thereby providing a consistent and uniform presentation of the sample for illumination and detection by the camera. Sheets of glass, Lucite® polycarbonate, flexible Mylar® polyester or other transparent material may be used to flatten the samples by layering the leaves between the transparent sheets. Depending on the type of analysis required, both surfaces of the host leaf can be easily imaged with such flattened samples. This aspect is particularly useful when imaging diseases that manifest infection randomly on each side of the leaf. For example, powdery mildew infection of cereals is highly variable between the two leaf surfaces, and both surfaces should be examined to provide an accurate assessment of the disease coverage. In contrast, the extent of tomato late blight disease is coincident on both the upper and lower surface of the leaf, and only one surface needs to be imaged for an accurate assessment of the degree of infection.

Fluorescence data are ordinarily acquired in the presence of the excitation illumination, whereas bioluminescence data are ordinarily acquired in the absence of external illumination. The integration times may be adjusted according to the intensity of the light signal, which is dependent upon the nature of the light generating moiety (e.g. fluorescence or bioluminescence), the spectral characteristics of the fluorescent light generating moiety (e.g. FDA, FP's or chlorophyll), and the pathology of the disease organism (e.g. surface vs. internal pathogen). A grayscale reference image of the sample can be acquired under white light using the macro imaging system.

In some applications of the methods, it may be desirable to localize the disease organism and/or express the growth or extent of the disease organism relative to the total area of the substrate, or leaf in the case of a host plant substrate. As an example of this aspect of the methods, the fractional area of a leaf infected with a disease organism containing a LGM is determined according to the following Flow Scheme. One skilled in the art can practice this method by operating the photon detectors to detect the photon emissions and analyze the images according to the Flow Scheme. One skilled in the art of computer programming can obtain or create programs to carry out these steps by using commercially available software or custom software.

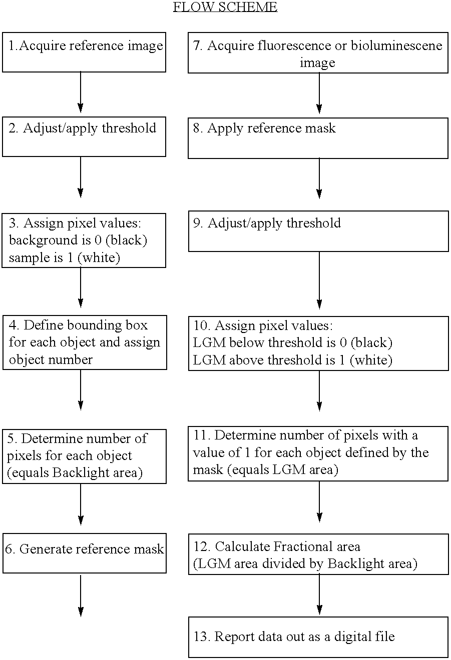

The example of image acquisition and analysis represented in the Flow Scheme is further described as follows. A reference image of the sample is acquired under dim white light illumination. A customized software application is used to threshold the image, such that each pixel above a set intensity threshold is assigned a value of 1 for the sample, and each pixel with an intensity below that threshold is identified as the background field of view and assigned a value of 0. This step generates a binary image that is subsequently used to determine the total area of each object in the field of view. The software application delineates a minimum bounding box around each contiguous object with pixel values of 1, and thereby defines each object in the field of view, and assigns it an object number. A minimum pixel number size can be set to eliminate detection and localization of extraneous objects (e.g. dirt or other debris) that may contaminate the field of view. The total number of objects in the field of view is thus defined, and the total number of pixels comprising each object, or the total leaf area, is determined.

The binary image is also used to generate a reference mask for the subsequent image acquisition step, i.e. detection of light that is generated by the fluorescent or bioluminescent moiety from regions of the defined object. The fluorescence or bioluminescence image is thresholded, assigning pixel values equal to 1 for regions of the object producing luminescence above a set threshold, and assigning pixel values of 0 to regions of the object with luminescence below that threshold. The pixels within the boundary of each object that contain a light generating moiety are thus defined, and the total number of pixels in which the light generating moiety is localized are determined.

To determine the fractional area containing the disease organism with a LGM, the number of pixels associated with localized light-generation is divided by the total number of pixels comprising the object or leaf. The image detecting the localization of the disease organism can be superimposed on the reference image of the plant substrate to form a composite image providing a spatial frame of reference. The composite image may be further analyzed to provide spatial information on the location and/or distribution of the disease organism. For example, the leaf may be divided into segments of a defined pixel length, and the fractional area of each segment, from the tip of the leaf to the base, infected by the disease organism calculated.

For other applications of the present invention, it may be desirable to forgo a detailed spatial determination of the fractional area of the substrate infected, and to instead determine the accumulated amount of disease organism present by integrating the total luminescence from a LGM within a defined sample area. Such instances arise, for example, when a fairly uniform area for the infection court is utilized, or when the configuration of the samples precludes the acquisition of images with sufficient spatial detail. For example, when the substrate is a microtitre plate well with or without other growth media or support, or a host plant within a microtitre plate well, or plants cultivated such that relatively uniform leaf area results, spatial information may become irrelevant. Similarly, when multiple hosts plants are contained within a confined area such as in a small horticultural pot or microtitre plate well, and/or when the axis of the photon detector is parallel to the long axis of cereal and grass plants, sufficient spatial information may be unattainable. As an example of this aspect of the methods, the detection and integration of the signal from multiple plants contained within a single well of a microtitre plate and infected with a disease organism containing a LGM is performed as follows.

An image of the sample fluorescence or bioluminescence, in this example for the entire microtitre plate, is acquired under excitation illumination. The operator can position a map of the plate that indexes and delimits the positions of the individual wells over the image of the plate on the computer monitor. The pixel intensities from this image are summed over the area defined for each well of the plate and the total signal from the fluorescent or bioluminescent LGM is reported out by well position. The luminescence intensity within the boundary of each well is thus defined, and is used as a measure of the accumulated amount of the disease organism.

All data output from the imaging system is digitized and easily input into a spreadsheet or data handling system of choice for further analysis and archiving. The fractional area of infection (i.e., percent disease) and/or the accumulated amount of the disease organism for experimental plant treatment agents are compared to the same measures for infected and untreated samples and infected samples treated with chemical standards at concentrations known to be efficacious in the control of plant disease.

VII. Production of Crop Protection Agents

One aspect of producing crop protection agents effective in the control of plant disease (e.g., fungicides, antimicrobial and antiviral agents, and/or inducers of systemic acquired disease resistance) is the procurement of plant treatment agents that are effective for controlling the plant disease organism that causes the disease. In some instances, plant treatment agents may be procured (when they are available) by purchase from manufacturers or other suppliers. The methods of this invention may be used to determine the effectiveness of the plant treatment agent by the supplier or by the party procuring it. In other instances, known plant treatment agents may be prepared by the producer of the crop protection agent. The methods of this invention may be used to determine the effectiveness of the plant treatment agent by the producer. In addition, new plant treatment agents that are particularly effective for controlling a plant disease may be discovered using the methods of this invention. The methods of this invention may be used in the production of crop protection agents by determining the effectiveness of plant treatment agents and compositions containing them for the control of plant disease organisms. The determination of effectiveness can represent an important step in the production of crop protection agents suitable for agronomic utility. For example, determining the effectiveness of a plant treatment agent not previously known to be effective in the control of plant disease organisms and/or compositions containing said plant treatment agent allows one skilled in the art to select said treatment agent and/or said compositions for production as crop protection agents. Determining the effectiveness of a previously unknown composition containing a previously known plant treatment agent allows one skilled in the art to select said composition for production as a crop protection agent. The methods of this invention may be used to determine the effectiveness of mixtures of plant treatment agents for controlling plant disease. One may also use methods of this invention to assay samples of known compositions to determine their effectiveness for controlling plant disease for quality control purposes during the manufacture, production and/or storage of said compositions.

Effective Plant Treatment Agents

Certain plant treatment agents determined to be effective by the methods of this invention are substances that may be used as active ingredients in the production of crop protection agents effective for the control of plant disease. They may be chemical compounds or mixtures of chemical compounds (e.g., a chemical mixture resulting from a physical mixing process or a mixed chemical synthesis process, a fermentation broth, or an extract preparation from a biological or non-biological origin). Chemical compounds determined to be effective can be obtained from chemical manufacturers and are typically prepared by those skilled in the art of chemical synthesis by chemical processes and transformations, including traditional solution-phase syntheses, syntheses employing combinatorial chemistry techniques such as polymer-bound or solid-phase reagents or substrates and parallel synthesis techniques and/or processes employing microbial agents, enzymes or enzyme preparations such as fermentations.

Formulation

Plant treatment agents will generally be used as crop protection agents consisting of a formulation or composition with an agriculturally suitable carrier comprising at least one of a liquid diluent, a solid diluent or a surfactant. Accordingly, another aspect of producing crop protection agents effective in the control of plant disease can involve formulating plant treatment agents with other components. The methods of this invention may be used to determine the effectiveness of the plant treatment agent in combination with the other components of a particular formulation. Normally, the formulation or composition ingredients are selected to be consistent with the physical properties of the active ingredient(s) (i.e., the plant treatment agent), mode of application and environmental factors such as soil type, moisture and temperature. Useful formulations include liquids such as solutions (including emulsifiable concentrates), suspensions, emulsions (including microemulsions and/or suspoemulsions) and the like which optionally can be thickened into gels. Useful formulations further include solids such as dusts, powders, granules, pellets, tablets, films, and the like which can be water-dispersible (“wettable”) or water-soluble. Active ingredient can be (micro)encapsulated and further formed into a suspension or solid formulation; alternatively the entire formulation of active ingredient can be encapsulated (or “overcoated”). Encapsulation can control or delay release of the active ingredient. Sprayable formulations can be extended in suitable media and used at spray volumes from about one to several hundred liters per hectare. High-strength compositions are primarily used as intermediates for further formulation.

The formulations will typically contain effective amounts of active ingredient(s) and diluent(s) and surfactant(s) within the following approximate ranges that add up to 100 percent by weight.

| | Weight Percent | | |
|---|---|---|---|
| | Active | | |
| | Ingredient(s) | Diluent(s) | Surfactant(s) |
| Water-Dispersible and | 5-90 | 0-94 | 1-15 |
| Water-soluble Granules, | | | |
| Tablets and Powders. | | | |
| Suspensions, Emulsions, | 5-50 | 40-95 | 0-15 |
| Solutions (including | | | |
| Emulsifiable Concentrates) | | | |
| Dusts | 1-25 | 70-99 | 0-5 |
| Granules and Pellets | 0.01-99 | 5-99.99 | 0-15 |
| High Strength Compositions | 90-99 | 0-10 | 0-2 |

Typical solid diluents are described in Watkins, et al., Handbook of Insecticide Dust Diluents and Carriers, 2nd Ed., Dorland Books, Caldwell, N.J. Typical liquid diluents are described in Marsden, Solvents Guide, 2nd Ed., Interscience, New York, 1950. McCutcheon's Detergents and Emulsifiers Annual, Allured Publ. Corp., Ridgewood, N.J., as well as Sisely and Wood, Encyclopedia of Surface Active Agents, Chemical Publ. Co., Inc., New York, 1964, list surfactants and recommended uses. All formulations can contain minor amounts of additives to reduce foam, caking, corrosion, microbiological growth and the like, or thickeners to increase viscosity.

Surfactants include, for example, polyethoxylated alcohols, polyethoxylated alkylphenols, polyethoxylated sorbitan fatty acid esters, dialkyl sulfosuccinates, alkyl sulfates, alkylbenzene sulfonates, organosilicones, N,N-dialkyltaurates, lignin sulfonates, naphthalene sulfonate formaldehyde condensates, polycarboxylates, and polyoxyethylene/polyoxypropylene block copolymers. Solid diluents include, for example, clays such as bentonite, montmorillonite, attapulgite and kaolin, starch, sugar, silica, talc, diatomaceous earth, urea, calcium carbonate, sodium carbonate and bicarbonate, and sodium sulfate. Liquid diluents include, for example, water, N,N-dimethylformamide, dimethyl sulfoxide, N-alkylpyrrolidone, ethylene glycol, polypropylene glycol, paraffins, alkylbenzenes, alkylnaphthalenes, oils of olive, castor, linseed, tung, sesame, corn, peanut, cotton-seed, soybean, rape-seed and coconut, fatty acid esters, ketones such as cyclohexanone, 2-heptanone, isophorone and 4-hydroxy-4-methyl-2-pentanone, and alcohols such as methanol, cyclohexanol, decanol and tetrahydrofurfuryl alcohol.

Solutions, including emulsifiable concentrates, can be prepared by simply mixing the ingredients. Dusts and powders can be prepared by blending and, usually, grinding as in a hammer mill or fluid-energy mill. Suspensions are usually prepared by wet-milling; see, for example, U.S. Pat. No. 3,060,084. Granules and pellets can be prepared by spraying the active material upon preformed granular carriers or by agglomeration techniques. See Browning, “Agglomeration”, Chemical Engineering, Dec. 4, 1967, pp 147-48, Perry's Chemical Engineer's Handbook, 4th Ed., McGraw-Hill, New York, 1963, pages 8-57 and following, and WO 91/13546. Pellets can be prepared as described in U.S. Pat. No. 4,172,714. Water-dispersible and water-soluble granules can be prepared as taught in U.S. Pat. No. 4,144,050, U.S. 3,920,442 and DE 3,246,493. Tablets can be prepared as taught in U.S. Pat. No. 5,180,587, U.S. 5,232,701 and U.S. 5,208,030. Films can be prepared as taught in GB 2,095,558 and U.S. Pat. No. 3,299,566.

For further information regarding the art of formulation, see U.S. Pat. No. 3,235,361, Col. 6, line 16 through Col. 7, line 19 and Examples 10-41; U.S. Pat. No. 3,309,192, Col. 5, line 43 through Col. 7, line 62 and Examples 8, 12, 15, 39, 41, 52, 53, 58, 132, 138-140, 162-164, 166, 167 and 169-182; U.S. Pat. No. 2,891,855, Col. 3, line 66 through Col. 5, line 17 and Examples 1-4; Klingman, Weed Control as a Science, John Wiley and Sons, Inc., New York, 1961, pp 81-96; and Hance et al., Weed Control Handbook, 8th Ed., Blackwell Scientific Publications, Oxford, 1989.

It is possible that some formulations containing a particular plant treatment agent may be more effective in controlling a plant disease than other formulations containing said plant treatment agent. The methods of this invention may be used to confirm the effectiveness of the plant treatment agent in a particular formulation.

In the following Examples of formulations, all percentages are by weight and all formulations are prepared in conventional ways using plant treatment agents (active ingredients) determined to be effective by the methods of this invention.

### EXAMPLE A

| Wettable Powder | |
|---|---|
| Active Ingredient(s) | 65.0% |
| dodecylphenol polyethylene glycol ether | 2.0% |
| sodium ligninsulfonate | 4.0% |
| sodium silicoaluminate | 6.0% |
| montmorillonite (calcined) | 23.0%. |

### EXAMPLE B

| Granule | |
|---|---|
| Active Ingredient(s) | 10.0% |
| attapulgite granules (low volatile matter, | 90.0% |
| 0.71/0.30 mm; U.S.S. No. 25-50 sieves). | |

### EXAMPLE C

| Extruded Pellet | |
|---|---|
| Active Ingredient(s) | 25.0% |
| anhydrous sodium sulfate | 10.0% |
| crude calcium ligninsulfonate | 5.0% |
| sodium alkylnaphthalenesulfonate | 1.0% |
| calcium/magnesium bentonite | 59.0%. |

### EXAMPLE D

| Emulsifiable Concentrate | |
|---|---|
| Active Ingredient(s) | 20.0% |
| blend of oil soluble sulfonates | 10.0% |
| and polyoxyethylene ethers | |
| isophorone | 70.0%. |

Mixtures

Plant treatment agents determined to be effective by the methods of this invention can also be mixed with one or more other insecticides, fungicides, nematocides, bactericides, acaricides, growth regulators, chemosterilants, semiochemicals, repellents, attractants, pheromones, feeding stimulants or other biologically active plant treatment agents to form a multi-component crop protection agent giving an even broader spectrum of agricultural protection. These mixtures may be prepared by obtaining the individual active ingredients and combining them with other formulation components as described above to provide a formulation containing two or more active ingredients. Mixtures may also be obtained by obtaining formulations containing individual active ingredients and physically mixing them to obtain a new formulation containing two or more active ingredients. The effectiveness of mixtures for controlling plant disease containing two or more active ingredients may be determined using the methods of this invention.

Examples of such agricultural protectants that can be included in compositions tested by the methods of this invention are: insecticides such as abamectin, acephate, azinphos-methyl, bifenthrin, buprofezin, carbofuran, chlorfenapyr, chlorpyrifos, chlorpyrifos-methyl, cyfluthrin, beta-cyfluthrin, cyhalothrin, lambda-cyhalothrin, deltamethrin, diafenthiuron, diazinon, diflubenzuron, dimethoate, esfenvalerate, fenoxycarb, fenpropathrin, fenvalerate, fipronil, flucythrinate, tau-fluvalinate, fonophos, imidacloprid, isofenphos, malathion, metaldehyde, methamidophos, methidathion, methomyl, methoprene, methoxychlor, methyl 7-chloro-2,5-dihydro-2-[[N-(methoxycarbonyl)-N-[4-(trifluoromethoxy)phenyl]amino]carbonyl]indeno[1,2-e][1,3,4]oxadiazine-4a(3H)-carboxylate (indoxacarb, DPX-JW062), monocrotophos, oxamyl, parathion, parathion-methyl, permethrin, phorate, phosalone, phosmet, phosphamidon, pirimicarb, profenofos, rotenone, sulprofos, tebufenozide, tefluthrin, terbufos, tetrachlorvinphos, thiodicarb, tralomethrin, trichlorfon and triflumuron; fungicides such as acibenzolar, azoxystrobin, benomyl, blasticidin-S, Bordeaux mixture (tribasic copper sulfate), bromuconazole, carpropamid (KTU 3616), captafol, captan, carbendazim, chloroneb, chlorothalonil, copper oxychloride, copper salts, cymoxanil, cyproconazole, cyprodinil (CGA 219417),(S)-3,5-dichloro-N-(3-chloro-1-ethyl-1-methyl-2-oxopropyl)-4-methylbenzamide (RH 7281), diclocymet (S-2900), diclomezine, dicloran, difenoconazole,(S)-3,5-dihydro-5-methyl-2-(methylthio)-5-phenyl-3-(phenylamino)-4H-imidazol-4-one (RP 407213), dimethomorph, diniconazole, diniconazole-M, dodine, edifenphos, epoxiconazole (BAS 480F), famoxadone, fenamidone, fenarimol, fenbuconazole, fencaramid (SZX0722), fenpiclonil, fenpropidin, fenpropimorph, fentin acetate, fentin hydroxide, fluazinam, fludioxonil, flumetover (RPA 403397), fluquinconazole, flusilazole, flutolanil, flutriafol, folpet, fosetyl-aluminum, furalaxyl, furametapyr (S-82658), hexaconazole, ipconazole, iprobenfos, iprodione, isoprothiolane, kasugamycin, kresoxim-methyl, mancozeb, maneb, mefenoxam, mepronil, metalaxyl, metconazole, metominostrobin/fenominostrobin (SSF-126), myclobutanil, neo-asozin (ferric methanearsonate), oxadixyl, penconazole, pencycuron, probenazole, prochloraz, propamocarb, propiconazole, pyraclostrobin, pyrifenox, pyrimethanil, pyroquilon, quinoxyfen, spiroxamine, sulfur, tebuconazole, tetraconazole, thiabendazole, thifluzamide, thiophanate-methyl, thiram, triadimefon, triadimenol, tricyclazole, trifloxystrobin, triticonazole, validamycin and vinclozolin; nematocides such as aldoxycarb and fenamiphos; bactericides such as streptomycin; acaricides such as amitraz, chinomethionat, chlorobenzilate, cyhexatin, dicofol, dienochlor, etoxazole, fenazaquin, fenbutatin oxide, fenpropathrin, fenpyroximate, hexythiazox, propargite, pyridaben and tebufenpyrad; and biological agents such as Bacillus thuringiensis, Bacillus thuringiensis delta endotoxin, baculovirus, and entomopathogenic bacteria, virus and fungi.

VIII. Methods for Controlling Plant Diseases

The present invention further provides a method for controlling plant diseases (e.g. a disease caused by a fungal plant pathogen) comprising determining that a plant treatment agent is effective by a method of this invention and applying to the plant or portion thereof to be protected, or to the plant seed or seedling to be protected, an effective amount of said plant treatment agent (e.g. as a component of a fungicidal composition containing said plant treatment agent). The methods of this invention may be used to identify plant treatment agents and compositions effective in providing control of diseases caused by one or more fungal plant pathogens in the Basidiomycete, Ascomycete, Oomycete and Deuteromycete classes. They may be effective in controlling plant diseases, particularly foliar pathogens of ornamental, vegetable, field, cereal, and fruit crops. These pathogens include Plasmopara viticola, Phytophthora infestans, Peronospora tabacina, Pseudoperonospora cubensis, Pythium aphanidermatum, Alternaria brassicae, Septoria nodorum, Septoria tritici, Cercosporidium personatum, Cercospora arachidicola, Pseudocercosporella herpotrichoides, Cercospora beticola, Botrytis cinerea, Monilinia fructicola, Pyricularia oryzae, Podosphaera leucotricha, Venturia inaequalis, Erysiphe graminis, Uncinula necatur, Puccinia recondita, Puccinia graminis, Hemileia vastatrix, Puccinia striiformis, Puccinia arachidis, Rhizoctonia solani, Sphaerotheca fuliginea, Fusarium oxysporum, Verticillium dahliae, Pythium aphanidermatum, Phytophthora megasperma, Sclerotinia sclerotiorum, Sclerotium rolfsii, Erysiphe polygoni, Pyrenophora teres, Gaeumannomyces graminis, Rynchosporium secalis, Fusarium roseum, Bremia lactucae and other genera and species closely related to these pathogens.

In certain instances, plant treatment agents that include a combination of one active component with another active component (see Mixtures in Section VII) having a similar spectrum of control but a different mode of action will be particularly advantageous for resistance management. Of note are plant treatment agents that are used for controlling fungal plant diseases by the methods of this invention and that comprise azoxystrobin, cymoxanil, epoxiconazole, famoxadone, fenamidone, fenpropimorph, flusilazole, fosetyl-aluminum, kresoxim-methyl, mancozeb, maneb, metalaxyl, metconazole, oxadixyl, pyraclostrobin, quinoxyfen, tricyclazole and/or trifloxystrobin in combination with another active ingredient of a different mode of action.

Plant disease control is ordinarily accomplished by applying an effective amount of a plant treatment agent of this invention either pre- or post-infection, to the portion of the plant to be protected such as the roots, stems, foliage, fruit, seeds, tubers or bulbs, or to the media (soil or sand) in which the plants to be protected are growing. The plant treatment agent can also be applied to the seed to protect the seed and seedling.

Rates of application for these crop protection agents can be influenced by many factors of the environment and should be determined under actual use conditions. Foliage can normally be protected when treated at a rate of from less than 1 g/ha to 5,000 g/ha of active ingredient. Seed and seedlings can normally be protected when seed is treated at a rate of from 0.1 to 10 g of active ingredient per kilogram of seed.

### EXAMPLES

A. Plant Cultivation, Treatment and Inoculation

Barley plants (Hordeum vulgari, cv. Boone) are cultivated either as single or multiple plants per plant growth unit, typically in a 2.54 cm by 2.54 cm pot of soil. Plants are grown in an environmentally regulated growth chamber at 20° C. and 70% humidity. The plants are approximately 7 days old at the time of application of the plant treatment agent. Single plant units are sprayed with fungicide standards or experimental plant treatment agents to the point of run-off.

Twenty-four hours following application of the plant treatment agent the barley plants are subsequently inoculated with spores of a fungal plant pathogen and incubated under the appropriate conditions for a period sufficient to allow the development of visible disease symptoms.

Inoculation with powdery mildew is with a spore suspension of Erysiphe graminis f. sp. hoerdei, (the causal agent of barley powdery mildew). Powdery mildew-inoculated plants are placed in a dry area for 4 hours following inoculation and are subsequently grown for an additional seven days at 20° C. and 65% humidity. Powdery mildew-infected plants are 15 days old at the time of rating of disease.

Barley blast inoculation is by spraying the plants with a spore suspension of Magnaportha grisea (the causal agent of blast disease). Either wild type or a transgenic strain of M. grisea, expressing a FP gene (ZsGreen FP) under the control of the ribosomal protein RP27 (p2) promoter (described below) are used for inoculation. Blast-inoculated barley plants are incubated in a saturated atmosphere at 27° C. for 24 h, and then moved to a growth chamber at 30° C. for 5 days, after which disease ratings are made. Blast-infected plants are 13 days old at the time of rating of disease.

Eragrostis curvula plants are cultivated in deep-well polystyrene microtitre plates containing solid ½× Murashige and Skoog (MS) media and covered with gas-permeable seals (Marsh Biomedical). Plants are seeded to approximately 5-6 seeds per well and are grown in an environmentally regulated growth chamber at 25° C. and 85% humidity, with a 1% CO₂ enriched atmosphere. Blast inoculation is performed when the plants are 10 days old by spraying the plants with a spore suspension of Magnaportha grisea. A transgenic strain of M. grisea, expressing an engineered variant of the GFP gene under the control of the ribosomal protein RP27 (p2) promoter (see FIG. 2 for a plasmid map) can be used for infection (see above for preparation of such a transgenic strain). The plates containing the blast-inoculated plants are subsequently covered with the gas permeable seals and maintained under conditions similar to that for growth of the host plants prior to infection. Disease ratings are made 4 days after inoculation. Fungicide standards or experimental plant treatment agents are applied as a systemic application to the media prior to seeding.

Tomato plants (Lycopersicon esculentum cv. Orange Pixie) are cultivated as single plants per plant growth unit, typically in a 2.54 cm by 2.54 cm pot of soil. Plants are grown in an environmentally regulated growth chamber at 27° C. and 70% humidity. The plants are approximately 14 days old at the time of application of the plant treatment agent. Single plant units are sprayed with fungicide standards or experimental plant treatment agents to the point of run-off.

Twenty-four hours following application of the plant treatment agent the tomato plants are subsequently inoculated with spores of a fungal plant pathogen, and incubated under the appropriate conditions for a period sufficient to allow the development of visible disease symptoms.

Inoculation with tomato late blight is with a spore suspension of Phytophthora infestans, (the causal agent of tomato late blight disease). Following inoculation, the plants are held for 24 hours in a saturating atmosphere and are subsequently grown for an additional seven days at 20° C. and 70% humidity. Late blight-infected tomato plants are 20 days old at the time of rating of disease.

Dried Sclerotinia sclerotiorum-infected dry bean blossoms used to infect the transgenic tobacco plants expressing the luciferase gene are generated as follows. Flower blossoms from 5-week old dry bean plants (Phaseolus vulgaris, ‘garden bush’ red kidney) are harvested and placed in a 150×15 mm petri dish containing moistened filter paper. Blossoms are inoculated with a Sclerotinia sclerotiorum spore suspension containing 20,000 spores/mL using a Preval® applicator. The petri dish containing the inoculated blossoms is sealed and stored in a 20° C. incubator for 48 hrs to initiate blossom infection. The resulting infected blossoms are allowed to air dry at 24° C. for 24 hrs before being stored at 4° C. in a sealed petri dish until use.

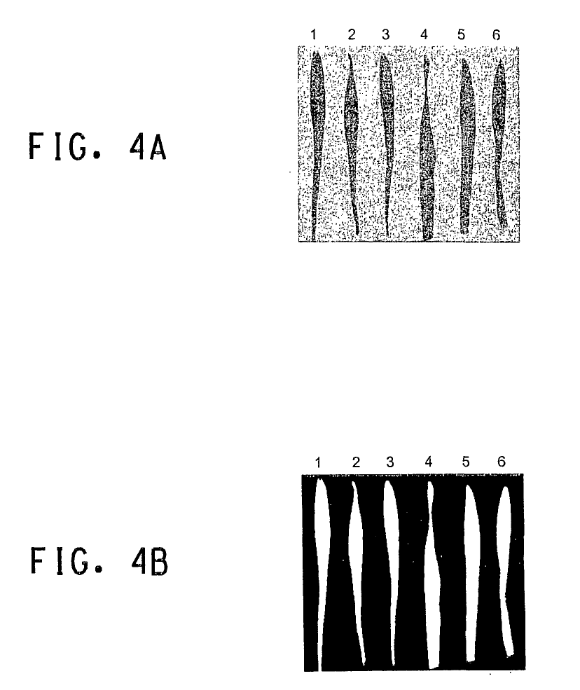

Tobacco seeds (Nicotiana tabacum cv. Xanthi), wildtype and 35S::LUC::NOS3′ T2 generation (see FIG. 3 for the plasmid map and section C below), are germinated on agar plates with 1×MS media containing 3% sucrose, and for transgenic lines 100 μg mL⁻¹ kanamycin and 100 μg mL⁻¹ carbenicillin in an environmentally regulated growth chamber at 24° C. Approximately 10-day-old seedlings are transferred to soil and grown under greenhouse conditions. Inoculation of 8-week old plants is by placing two dried Sclerotinia sclerotiorum-infected dry bean blossoms onto each pre-wetted tobacco leaf. Plants are moved to a 20° C. dew chamber for 48 hrs to promote leaf infection, after which time the plants are imaged.

B. Construction of the 35S::Luciferase Transgenic Tobacco

A schematic of the pSLA2 plasmid is represented in FIG. 3. The plasmid is constructed by cloning an 1.4 kb Bain HI/Nco I fragment containing the promoter sequences from the cauliflower mosaic virus (CaMV) 35S gene from the plasmid pML089 into the plasmid pSLA1, fused to the firefly luciferase gene, LUC, (Promega Corp., Madison, Wis.) followed by the NOS3′ poly-A addition sequence in a pKS II⁻ Bluescript plasmid (Stratagene, La Jolla, Calif.). The p35S::LUC::NOS3 ′ reporter gene is subcloned as a ˜3.3 kb Bam HI/Sal I fragment into the binary vector pZBL1N to generate pSLA2. The plasmid structure is confirmed by restriction enzyme digestion and size mapping of the fragments following electrophoresis and all cloning junctions are confirmed by sequencing using primers whose design is based on the sequences flanking the restriction enzyme cloning sites.

The binary plasmid pSLA2 is mobilized into the Agrobacterium strain LBA4404 by transformation of competent stocks of the bacterium according to standard protocols (in An et al., 1988, Binary vectors, in Plant Molecular Biology Manual, A3 (Gelvin, S. and Schilperoort, R. Eds), Dordrecht: Kluwer Academic Publishers). Plasmid structure in the transformed agrobacterium lines is confirmed by restriction digest analysis of plasmids isolated from liquid bacterial cultures using a modification of the Wizard Minipreps Purification System (Promega Corp, Madison, Wis.). Leaf segments of tobacco plants maintained in sterile culture on solid MS media containing 3% sucrose are transformed according to standard techniques (Horsch et al., 1988, Leaf disc transformation, in Plant Molecular Biology Manual, A5 (Gelvin, S. and Schilperoort, R. eds), Dordrecht: Kluwer Academic Publishers). Transformed callus tissue is selected on solid MS media containing 200 μg mL⁻¹ kanamycin and 500 μg mL⁻¹ carbenicillin. Regenerated shoots are selected on solid MS containing 3% sucrose, 100 μg mL⁻¹ kanamycin and 500 μg mL⁻¹ carbenicillin. Resistant T1 (primary transformant) plants carrying the 35S::LUC::NOS3′ fusion are subsequently grown to maturity in soil under greenhouse conditions and T2 seed collected.

C. Construction of the ZsGreen FP Expressing Magnaportha grisea Strain MG619

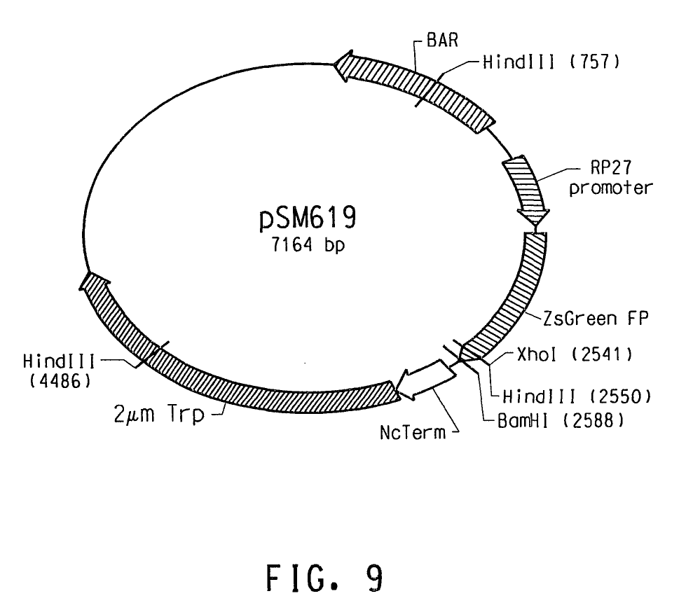

Construction of a FP-expressing Magnaportha grisea strain is carried out as described in the following. A series of plasmids referred to as “compro” (because all the plasmids in the series have a common 20 bp sequence at the 3′ end of the promoter) is constructed according to standard molecular biology techniques well-known to those skilled in the art. Such plasmids are based on pBluescript and contain a fungal promoter with the “compro” sequence, the Neurospora crassa B-tubulin transcription terminator, the bialaphos resistance gene for fungal selection, trp1 of Saccharomyces cerevisiae, and a 2 μm yeast origin of replication. A version containing the M. grisea RP27 (or P2) ribosomal protein promoter is pSM324. Plasmid pSM324 is digested with Xho I and the 5′ overhang filled-in with the Klenow fragment of DNA polymerase I. Of use in the present invention are FP's and their variants engineered for enhanced fluorescent yield relative to wildtype GFP, such as EGFP, ZsGreenFP (both available from Clontech Laboratories, Inc. Palo Alto, Calif.) and rsGFP (available from Quantum Biotechnologics, Inc. Montreal, Quebec, Canada). To generate a FP expression vector, polymerase chain reaction is used to amplify the FP coding sequences with 5′ and 3′ primers designed to the respective ends of the FP sequence and with sequence extensions homologous to the pSM324 sequence flanking the Xho I site and sufficient for homologous recombination. The primers “Zsgr5” (AGGAACCCAATCTTCAAAATGGCCCAGTCCAAGCAC) and “Zsgr3” (AATGTTGAGTGGAATGATTTATCTAGATCCGGTGG), designed to the Clontech ZsGreen FP sequence, are used for construction of a vector for expression of this PP in M. grisea. The Zsgr5/Zsgr3 amplification product and the Xho-digested pSM324 are then transformed into Saccharomyces cerevisiase strain W303-1A by the lithium acetate method (Agatep et al., 1998) to allow gap repair of the plasmid. Yeast colonies are selected on tryptophan-minus plates. Plasmid minipreps are performed on yeast tryptophan prototrophs and an aliquot transformed into E. coli strain DH10B by electroporation. Plasmid DNA is prepared from ampicillin-resistant E. coli transformants and screened by restriction digest for the proper gap repair of the Zsgr5/Zsgr3 amplification product into pSM324 to generate the FP expression vector pSM619 (FIG. 9). The FP expression vector pSM619 is transformed into M. grisea strain 4091-5-8 using published transformation protocols (Sweigard et al., 1992). Bialaphos-resistant transformants of M grisea are selected and purified by single spore selection. Independent transformants are screened for fluorescent intensity and an exceptionally bright transformant, MG619, is selected for further use.

D. Imaging: Fluorescence

Samples or objects to be imaged for fluorescence are placed in a light-tight imaging chamber containing a door, excitation lights, white lights for reference image acquisition, and a cooled CCD camera outfitted with a Nikkor® AF 35 mm f2 lens (Nikon Inc., Melville, N.Y.) and a suitable emission filter. Image data is obtained using a SenSys® 1401E camera system, available from Roper Scientific (Tucson, Ariz.). (See FIG. 1).

Since many of the plant samples have undergone significant growth since the time of treatment and infection, elaborating 2-3 new leaves, steps are taken to eliminate the new growth from the sample to be analyzed. The human scorer typically folds or holds the new growth out of the field of view when rating conventionally. To achieve the same level of discrimination with the imaging application of the present invention, the new growth is removed by physically cutting it off with scissors just prior to placement in the detection field. Alternatively, a software application may be used to define a region of interest in the image, eliminating the need for the physical removal of the new growth.

Reference images are obtained with the sample under dim white light illumination and are generated by integrating photons for a selected period of time, typically 100 msec. The threshold for pixel intensity is adjusted to discriminate between pixels associated with sample or the background, typically at a pixel intensity value of 60, in order to generate a binary image which defined each object in the field of view and its respective area in pixels.

Fluorescence data is obtained in the presence of the excitation light. For imaging of FDA-derived and green fluorescent protein-derived fluorescence (e.g. GFP- or ZsGreen FP-derived fluorescence) a XF1073 filter with Optical Density (OD) 5 blocking and 475 nm and 40 nm full width at half maximum transmission (FWHW) is used for excitation. An enhanced XF3084 filter with an AELP (alpha epsilon longpass) edge, 535 nm center wavelength (CWL) and 45 nm FWHW is used for the emission. For imaging of chlorophyll fluorescence a 430DF40 filter with OD 5 blocking and 430 nm CWL and 40 nm FWHW is used for excitation. A 600 AGLP (alpha gamma longpass) filter is used for the emission. All filters are obtained from Omega Optical, Inc., Brattleboro, Vt. Images are generated by integrating photons for a selected period of time, typically 2 sec. The threshold for pixel intensity is adjusted to discriminate between pixels associated with the LGM and the sample background, typically to a value of 60.

For the determination of the % disease area for a sample, a binary image is generated which defined the area of pixels associated with a LGM for each object in the field of view. The percent disease area is calculated by dividing the pixel number for the LGM area by the pixel number for the sample area. For the detection of a LGM endogenous to the plant substrate, an additional step of subtracting the fluorescence area from the total leaf area and then dividing by the total leaf area is required for the percent disease area calculation.

For the determination of the accumulated amount of disease organism by integrating the fluorescent signal, the samples were imaged using a Hamamatsu C2400 photon detector system outfitted with the excitation light source and emission filters described above for use with the SenSys camera system. Typically, the discriminator is set to 20, the sensitivity (camera gain) is set to 1 and the fluorescence signal is integrated for 10 sec. The fluorescence signal intensities of each pixel in the fluorescence image is summed for a defined region of interest, typically the area defined by the well of a microtitre plate, and is reported on a unit area basis. A reference image is collected under dim green light illumination by integrating typically 64 frames.

The reference and fluorescence image data may be obtained as grayscale images. In some instances, the reference and fluorescence images may be superimposed, using the image processor, to form a composite image. A hardcopy of the composite image is generated by saving the image as a digital file, transferring the file to the computer, and printing it on a printer attached to the computer.

E. Imaging: Bioluminescence

Samples or objects to be imaged for bioluminescence may be collected using the system described above employing the SenSys® detector provided that sufficient bioluminescence is produced by the sample.

Reference images of the sample are obtained under epi-illumination (i.e., illumination directed to the upper surface of the leaf) with dim white light and collected using the Sensys® detector. Bioluminescence data is obtained with the same detector in the absence of extraneous light.

The reference and bioluminescence image data may be obtained as grayscale and binary images. Hardcopies of the images are generated by saving the images as digital files, transferring the files to the computer, and printing them on a printer attached to the computer.

### Example 1

### Detecting Photon Emission from a Disease Organism Treated with a Pro-LGM and Infecting a Plant Substrate (Embodiment I)—Determination of Disease Area

The effectiveness of a plant treatment agent on plants infected with a disease organism is determined by treating the test unit with a pro-LGM and detecting photon emission. Barley plants cultivated as described above are given 125 μL spray applications of a preventative treatment containing 40, 5, 1, 0.4 and 0.2 ppm flusilazole. The plant treatment agent is formulated by resuspending the appropriate amount of the dried compound in 6 μL of DMSO with shaking overnight, followed by the subsequent addition of 570 μL of a 50/50 Acetone/Water-Trem solution, yielding the desired final concentration. The Water-Trem contains 18 drops of the Trem® 014 surfactant (Henkel Corp, Amber, Pa.) per L of water. The formulated plant treatment agent is applied as a foliar spray application. In this example, a microsprayer apparatus (U.S. Patent Application No. 60/172,928) applying a fine aerosol mist from the tip of an ultrasonic nebulizer, with air assist, to a rotating electrostatically charged perpendicular plant target is employed.

Twenty-four hours following application of the plant treatment agent the barley plants are subsequently inoculated with a suspension of powdery mildew (Erysiphe graminis f sp. hordei) spores, and incubated under the appropriate conditions for a period sufficient to allow the development of visible disease symptoms, as described above. Untreated plants are also infected at this time for the production of untreated, infected controls. Untreated, uninfected control plants are also generated in parallel.

Plants to be rated for fungicide activity are first scored visually by eye for % disease coverage. The same samples are subsequently treated with the pro-LGM fluorescein diacetate. The FDA is formulated in a stock solution at 1 mg FDA/mL acetone. A final working solution is formulated which contains 5 μg/mL fluorescein diacetate and 0.005% Trem® 014. The FDA working solution is applied by spraying the sample using a Preval® sprayer (Precision Valve Corporation, Yonkers, N.Y.) with sufficient solution to wet all surfaces. Care must be taken not to allow access of FDA to leaves with cuts or abraded surfaces. Such breaches of the waxy leaf cuticle allow FDA access to the xylem stream of the leaf vasculature system, and the entire leaf will be quickly labeled with FDA, preventing the differential staining of the pathogen with the LGM. After allowing sufficient time for the uptake and conversion of FDA to fluorescein, typically 5 min, the plant tissue that has grown after treatment and infection is physically removed and the samples are placed in the dark box of the imaging system. Reference and fluorescence image data is collected as described above, and processed for the determination of disease area. The image collection process and analysis is repeated sequentially ten times in order to evaluate the reproducibility of the determination made on the basis of photon emission.

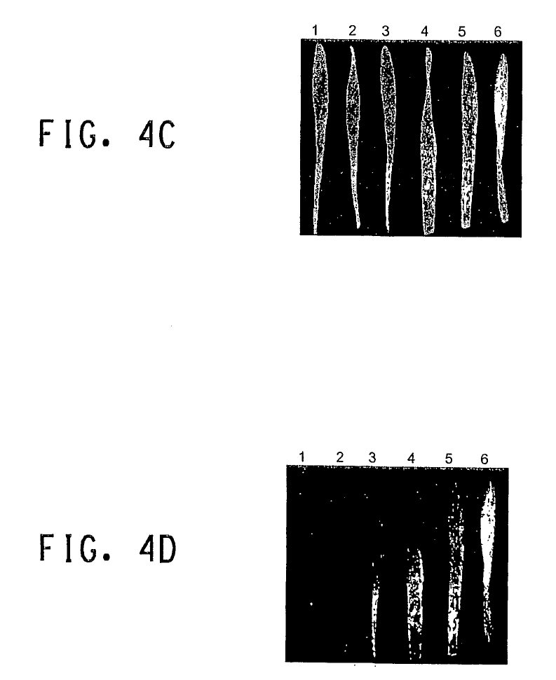

Grayscale and binary images of the reference and fluorescence data are presented in FIG. 4. FIGS. 4A and 4B represent the grayscale and binary images, respectively, of the plant samples collected under white light reference illumination, and FIGS. 4C and 4D represent the grayscale and binary images, respectively, of the same materials collected under fluorescence excitation conditions. Plant 1 is an untreated, uninfected control. Plants 2-6 are treated with 40, 5, 1, 0.4 and 0.2 ppm flusilazole, respectively, prior to infection. A comparison of % disease area obtained from visual scoring or based on photon emission is represented in Chart 1. Plants scored by eye are rated on a 0 to 5 scale in 0.5 unit increments. A score of 5 corresponds to 52% disease area, the maximum powdery mildew disease area assigned by visual scoring. As illustrated in the chart, data from both the fluorescence and visual scoring methods yield comparable results, but the determination of disease area by detection of photon emission by fluorescence is more sensitive than the visual scoring method.

Chart 1
% Disease Area

| Column No. | 1 | 2 | 3 | 4 | 5 | 6 |
|---|---|---|---|---|---|---|
| Treatment | Untreated | 40 ppm | 5 ppm | 1 ppm | 0.4 ppm | 0.2 ppm |
| | Uninfected | flusilazole | flusilazole | flusilazole | flusilazole | flusilazole |
| Visual | 0 | 0 | 1 | 7 | 19 | 52 |
| Scoring | | | | | | |
| Photon | 0 | 0 | 13 | 24 | 27 | 64 |
| Imaging | | | | | | |

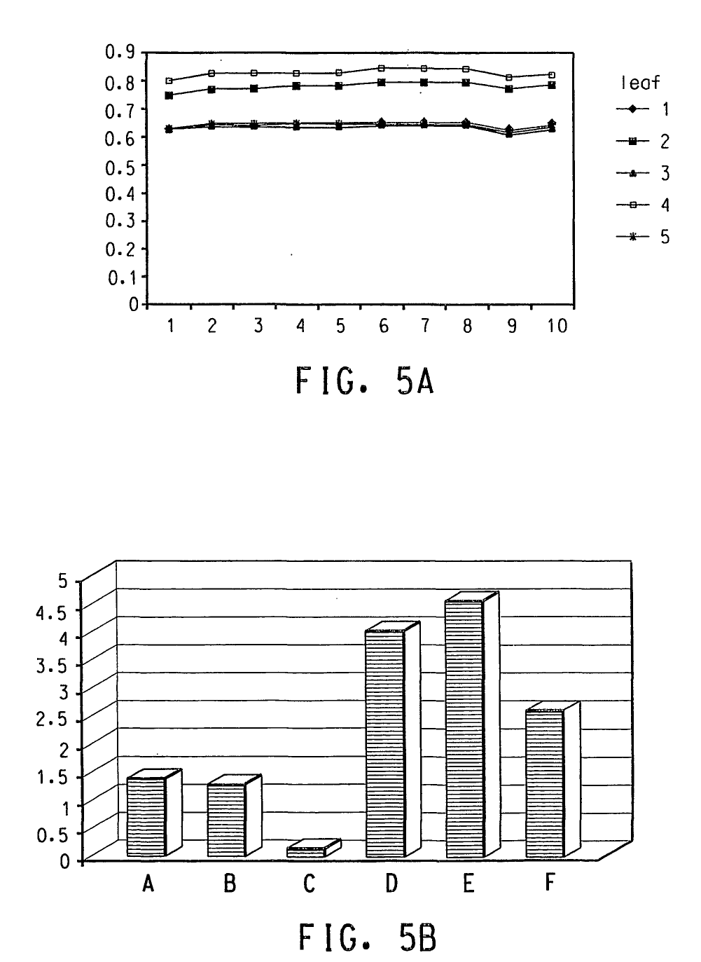

Data on the reproducibility of the measurements based on photon emission are presented in FIG. 5. FIG. 5A represents the disease area as a fraction of leaf surface (on the Y-axis) that are determined by imaging for 5 leaf samples of Erysiphae graminis infected on barley and that have been rated by eye to have 52% disease area. The samples are made fluorescent by differential staining with the PLGM fluorescein diacetate. Each sample is placed in the imaging chamber a single time and imaged 10 times sequentially (on the X-axis). As represented in FIG. 5A, the disease measurements for the 5 samples as determined by imaging range from a mean of 64 to 84% (compared to 52% for visual scoring), but the values are highly reproducible for each of the 5 samples upon repeated measurement. FIG. 5B represents the variability (Y-axis) in the % Disease area (A), LGM area (B) and Leaf area (C) measurements for the instrument itself (measured as in 5A) and variability in the % Disease area (D), LGM area (E) and Leaf area (F) measurements under typical operating conditions (to determine the variability under typical operating conditions the same samples are placed in the imaging chamber 10 times sequentially and imaged each time). The percent variation in the disease area determination attributable to the instrument alone is approximately 1.25%. Most of this variation arises from the measurement of the LGM area (approximately 1%), which is expected since photon emission is a stochastic process. The percent variation for the disease area determination increases to approximately 4% under typical operating conditions, reflecting variability in sample placement in the imaging chamber and in orientation relative to the camera. From these data, the precision of photon imaging is estimated to have a variability of approximately 4%. Thus, the imaging method of the present invention provides a robust and highly statistically-reproducible measure of the % disease area for determining the efficacy of a plant treatment agent even under typical operating conditions.

### Example 2

### Detecting Photon Emission from a Disease Organism Treated with a Pro-LGM (Embodiment I)—Integration of Photon Emission and Determination of Disease Organism Accumulation

The effectiveness of a plant treatment agent on a disease organism grown in liquid culture in wells of a microtitre plate is determined by treating the substrate (i.e., growth media contained within the microtitre plate well) with a pro-LGM and detecting photon emission.

Magnaportha grisea is cultured in 200 μL per well of liquid growth media and plant treatment agent (if any) contained within wells of a 96 well microtitre plate. The composition of the basal salt medium for growth of M. grisea is (per liter): 3.0 g K₂HPO₄, 4.0 g KH₂PO₄, 0.5 g NaCl, 1.0 g NH₄Cl, 0.2 g MgSO₄.7H₂O, 0.01 g CaCl₂.H₂O, 1 μg MnSO₄.H₂O, 2 μg ZnSO₄.7H₂O, 2 μg CuSO₄.5H₂O, 0.2 μg FeSO₄.7H₂O, 1 μg Na₂MO₄.2H₂O, 0.6 μg CoSO₄, 0.8 μg H₃BO₃, 0.01 μg biotin, 20 g glucose, and 50 μL Tween® 20 surfactant. The plant treatment agents are combined with the growth media to achieve the desired final concentration. In this test, Column 1 is comprised of controls treated with the DMSO diluent alone. Columns 2-6 are treated, respectively, with 1:3 serial dilutions of the following (with the starting concentration in ppm indicated): MBC (5), flusilazole (2), famoxadone (2), azoxystrobin (2) and captan (5). The same treatments are repeated in columns 7-12. The wells are inoculated with 200 μL of a M. grisea spore suspension at a concentration of 75,000 spores/mL, in the media described above. Cultures are maintained in the dark at 22° C. The fungal culture is grown for 7 days.

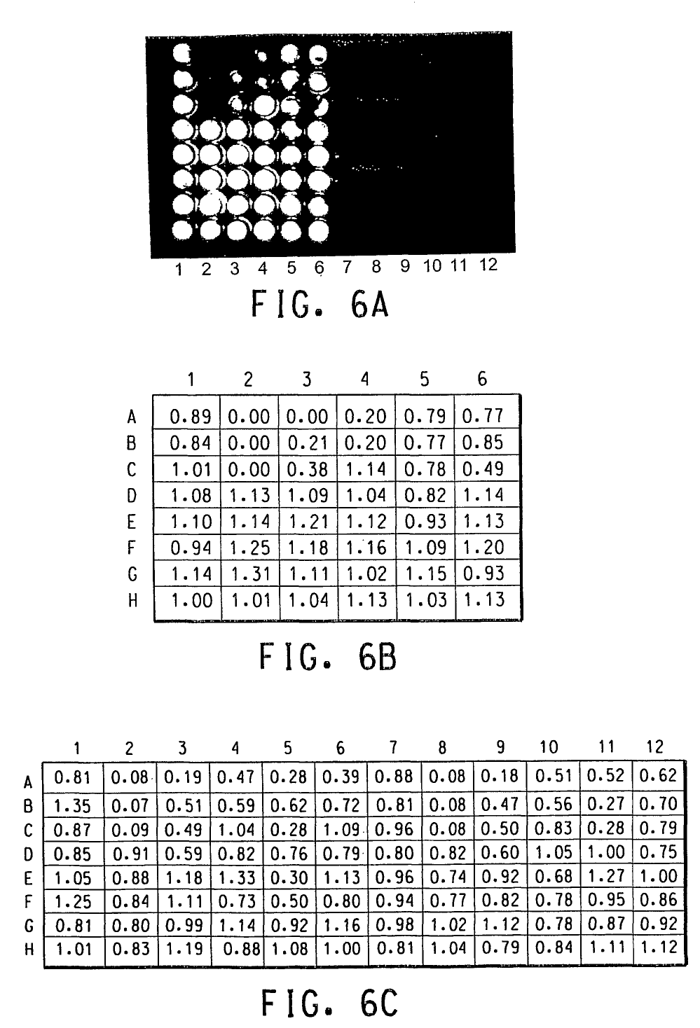

Plates to be rated for fungicide activity are first rated conventionally using a plate reader to measure optical density (OD) at 650 nm (see FIG. 6C). The samples are subsequently treated with the pro-LGM fluorescein diacetate (FDA). The FDA formulated as in EXAMPLE 1 above is applied in this example by pipeting 20 μL per well, or may also be applied by spraying the microtitre plate with sufficient solution to treat all the desired wells (wells not desired to be treated may be masked or the spray directed to prevent treatment of those wells). Columns 1-6 are treated with FDA and columns 7-12 are not treated with FDA. After allowing sufficient time for the uptake and conversion of FDA to fluorescein, typically 5 min, the samples are placed in the imaging chamber of the imaging system. Reference and fluorescence image data is collected as described above using a Hamamatsu C2400 photon detector (see above), and processed for the photon emission from each well.

A grayscale image of the fluorescence data is presented in FIG. 6A. The normalized photon counts are represented in FIG. 6B for columns 1-6. Photon counts for columns 7-12 are read to be zero and are not tabulated. FIG. 6C represents the normalized OD data at 650 nm. Both the photon counts and OD 650 nm data have been normalized relative to the mean values for the respective measures for the controls in column 1, allowing easier comparison of the data from the two measurement types. Comparison of the photon counts and the OD measurements indicate that the methods yield comparable results.

### Example 3

### Detecting Photon Emission from a Disease Organism Containing an Endogenous LGM and Infecting a Plant Substrate (Embodiment II)—Determination of Disease Area

The photon emission from an LGM endogenous to the disease organism infected on plants is detected as follows. Barley plants are inoculated with a suspension of spores of a transgenic strain of Magnaportha grisea expressing a FP or FP variant (e.g. GFP) engineered for enhanced fluorescence yield and incubated under the appropriate conditions for a period sufficient to allow the development of visible disease symptoms, as described above. Controls of uninfected plants and plants infected with the wildtype M. grisea are generated at the same time. The infected leaves are physically removed by clipping them from the plants and the samples are subsequently imaged for FP fluorescence. Reference and fluorescence image data is collected as described above.

The fluorescence from the FP M. grisea strain is easily detected above the background fluorescence of the endogenous plant LGMs induced upon infection with the wild-type pathogen.

### Example 4

### Detecting Photon Emission from a Disease Organism Containing an Endogenous LGM and Infecting a Plant Substrate (Embodiment II)—Integration of Photon Emission and Determination of Disease Accumulation

The effectiveness of a plant treatment agent on plants infected with a disease organism is determined by integrating photon emission from an LGM endogenous to the disease organism and determining the disease accumulation. This example illustrates quantifying fungal disease by measuring endogenous fluorescence under circumstances where ordinary visual rating is not practicable.

To demonstrate an example of the ability to detect an effective plant treatment agent using the present method, Eragrostis curvula plants are grown as described above in a deep well microtitre plate. A systemic application (wells with media pre-treated with the plant treatment agent prior to seeding) of a preventative treatment containing 5 ppm of the commercial fungicide tricyclazole is made in all columns of the plate except two columns, which serve as untreated, inoculated controls. The plants are inoculated with a spore suspension of a M. grisea strain expressing a FP (e.g. GFP variant engineered for enhanced fluorescence yield) and are incubated, as described above. The plate is imaged for FP fluorescence as described above. Plants in wells with media pre-treated with tricyclazole prior to seeding exhibit reduced fluorescence relative to the fluorescence observed with the untreated, inoculated control columns, correlating with reduced disease symptoms. Visual rating for quantitation is not possible with this test format because over-seeding of the samples and the vertical growth habit of the plants within the wells prevents scoring by eye. The detection of fluorescence photon emission provides a sensitive means of detecting control with a commercial fungicide effective on this pathogen. The ability to detect and rate fungicidal activity on a whole plant test in a plate format provides several potential advantages. Such a test maintains the ability to detect disruption of the host-pathogen interaction, reduces the compound requirement, may be automated and may provide increased assay throughput.

### Example 5

### Detecting Photon Emission from an Endogenous Plant LGM Localized in the Uninfected Region of a Plant Substrate (Embodiment II—Determination of Disease Area

The effectiveness of a plant treatment agent, on plants infected with a disease organism is determined by detecting photon emission from an LGM endogenous to the plant substrate. Tomato plants cultivated as described above are given 125 μL spray applications of a preventative treatment containing 20, 5, 1 and 0 ppm ridomil. The plant treatment agent is formulated and applied as described in EXAMPLE 1 above.

Twenty-four hours following application of the plant treatment agent the tomato plants are subsequently inoculated with a suspension of spores of Phytophthora infestans, the causative agent of tomato late blight, and incubated under the appropriate conditions for a period sufficient to allow the development of visible disease symptoms, as described above. Untreated, uninfected control plants are also generated in parallel.

Plants to be rated for fungicide activity are first scored conventionally by eye for % disease coverage. Plant tissue that has grown after treatment and inoculation is then physically removed and the samples are subsequently imaged for chlorophyll fluorescence. Reference and fluorescence image data is collected as described above, and processed for the determination of disease area.

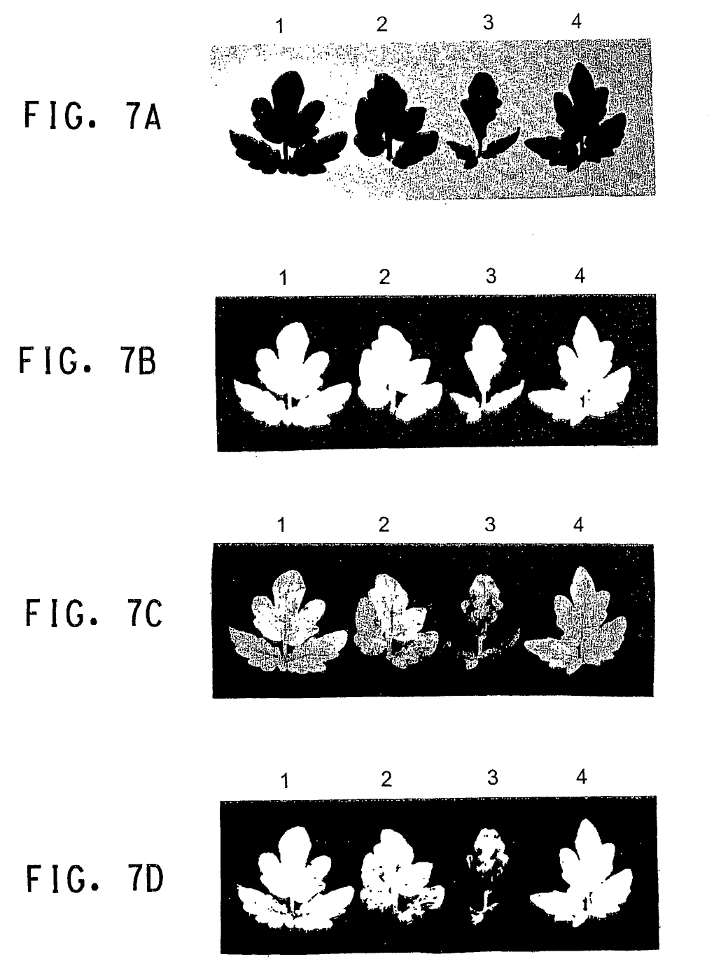

Grayscale and binary images of the reference and fluorescence data are represented in FIG. 7. Columns 1-3 represent images of leaves treated with 20, 5 and 1 ppm of ridomil and inoculated with P. infestans. Column 4 is an uninoculated and untreated leaf. Leaves inoculated with P. infestans and left untreated suffer severe necrosis and the leaves often abscise (not illustrated). FIGS. 7A and 7B represent the grayscale and binary images, respectively, of the plant samples collected under white light reference illumination, and FIGS. 7C and 7D represent the grayscale and binary images, respectively, of the same materials collected under fluorescence excitation conditions. A comparison of % disease area obtained from visual scoring or based on photon emission is represented in Chart 2. Data from both the fluorescence and visual scoring methods indicate that detection of photon emission and visual rating yield comparable results.

Chart 2
% Disease Area

| Column No. | 1 | 2 | 3 | 4 |
|---|---|---|---|---|
| Treatment | 20 ppm Ridomil | 5 ppm | 1 ppm | Uninoculated |
| | | Ridomil | Ridomil | |
| Visual Scoring | 3 | 24 | 53 | 0 |
| Photon Imaging | 2 | 10 | 60 | 2 |

### Example 6

### Detecting Photon Emission from the Uninfected Regions of a Plant Substrate Engineered to Express an Endogenous LGM (Embodiment II—Determination of Disease Area

The photon emission from a plant engineered to express an endogenous LGM and infected with a disease organism is detected as follows. Single leaves on 8 week 35S::LUC::NOS3′ tobacco plants produced and cultivated as described above are inoculated at two sites with Sclerotinia sclerotiorum, and incubated under the appropriate conditions for a period sufficient to allow the development of visible disease symptoms, as described above. Uninoculated control plants are also generated in parallel.

The samples are subsequently imaged for luciferase bioluminescence following the application of firefly luciferin, the substrate for bioluminescence. Firefly luciferin is available as the potassium salt from Promega Corp, Madison, Wis. The infected and control leaves of the plants are sprayed 3 times over a 2 hr period to run-off with an aqueous 5 mM luciferin solution containing 0.01% Triton X-100® surfactant. After allowing sufficient time for luciferin uptake, typically 10 min following the last luciferin application, individual leaves from plants are removed and placed inside the imaging chamber. Reference and bioluminescence image data is collected as described above. A second reference image may be obtained under backlight illumination with a flatbed light source (see FIG. 1, element 15) (e.g. available from Schott-Fostec, Auburn, N.Y.). The backlight image provides spatial reference for the location of the Sclerotinia infection zones, which are not evident under epi-illumination.

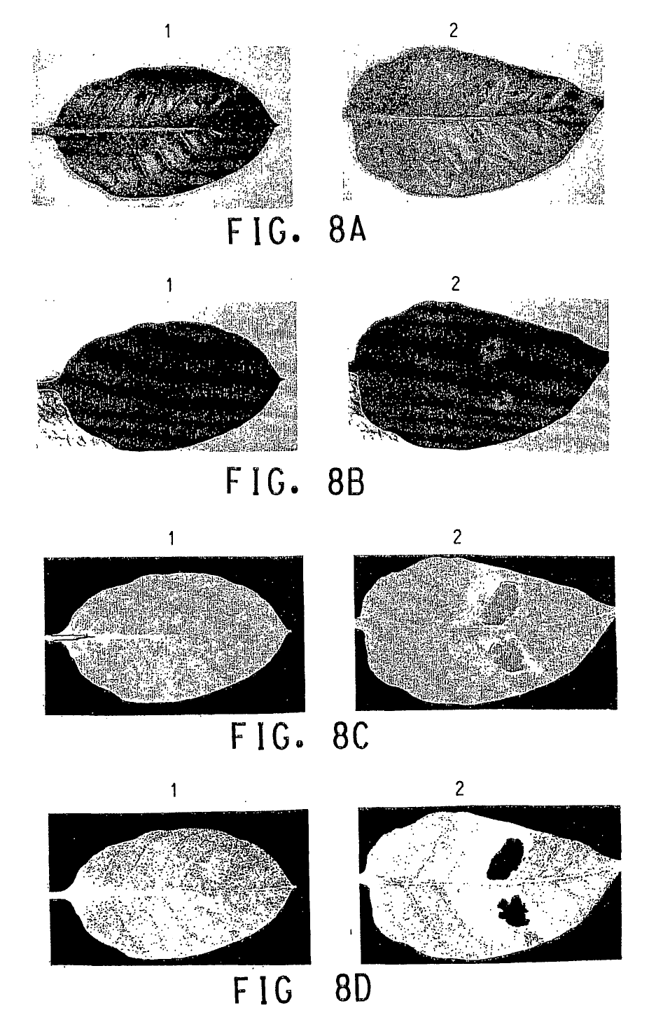

Grayscale and binary images of the bioluminescence are presented in FIG. 8. Column 1 represents uninoculated 35S::LUC::NOS3′ tobacco leaves and column 2 represents 35S::LUC::NOS3′ tobacco leaves inoculated with Sclerotinia. Row A represents a reference image collected under epi-illumination, row B represents the same leaves under backlight illumination, row C represents a grayscale image of the bioluminescence and row D represents a binary image. Areas of the tobacco plant infected with the pathogen are not bioluminescent and are readily detected by the imaging system.

### Example 7

### Detecting Photon Emission from a Disease Organism Engineered to Express an Endogenous LGM (Embodiment II)—Integration of Photon Emission and Determination of Disease Organism Accumulation

The effectiveness of a plant treatment agent on a disease organism engineered to express an endogenous LGM and grown in liquid culture in wells of a microtitre plate is determined by integrating photon emission and determining the disease accumulation. Transgenic Magnaportha grisea engineered to express a FP (e.g. GFP) as described above is cultured in liquid growth media and treated as above in Example 2 contained within wells of a 96 well microtitre plate. The samples are subsequently placed in the dark box of the imaging system. Reference and FP fluorescence image data is collected as described above, and processed for the photon emission from each well.

### Example 8

### Detecting Photon Emission from a Disease Organism Containing an Endogenous LGM and Infecting a Plant Substrate (Embodiment II)—Determination of Disease Area

The photon emission from an LGM endogenous to the disease organism infected on plants is detected as follows. Barley plants are inoculated with a suspension of spores of a transgenic strain (MG619) of Magnaportha grisea expressing a the ZsGreen FP and incubated under the appropriate conditions for a period sufficient to allow the development of visible disease symptoms, as described above. Controls of uninfected plants and plants infected with the wildtype M. grisea are generated at the same time. The infected leaves are physically removed by clipping them from the plants and the samples are subsequently imaged for ZsGreen FP fluorescence. Reference and fluorescence image data is collected as described above.

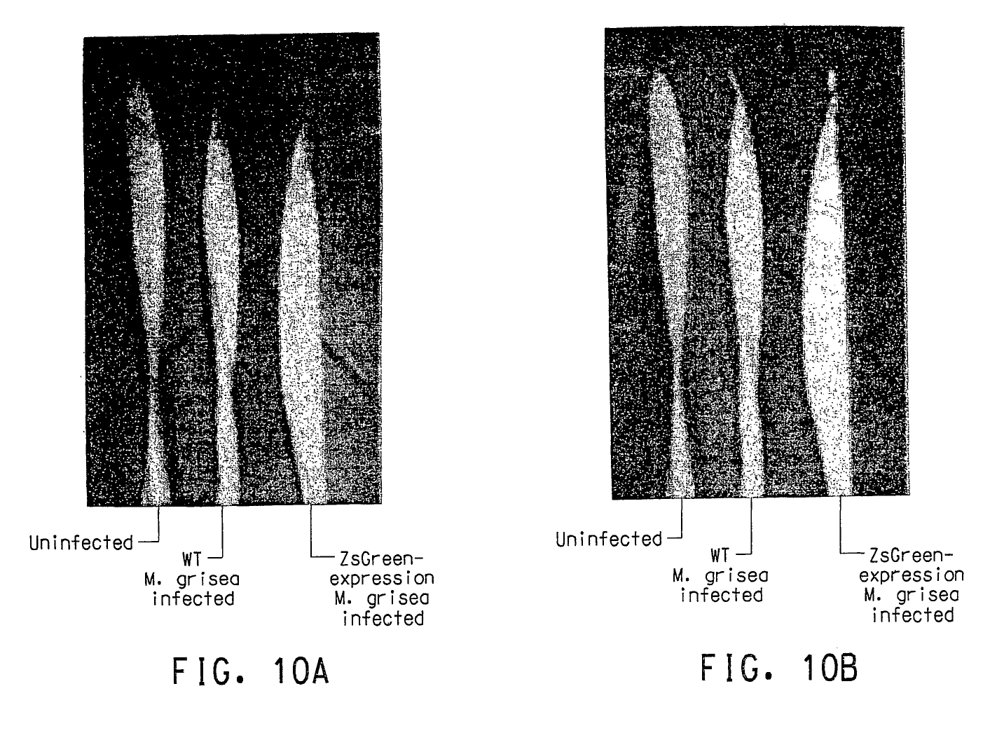

Reference grayscale and fluorescence grayscale images are represented in FIGS. 10A and B, respectively. Column A represents an uninoculated leaf, column B represents a leaf inoculated with wild type M. grisea, and column C represents a leaf inoculated with M. grisea expressing the ZsGreen FP (strain MG619). The fluorescence from the ZsGreen FP M. grisea strain is easily detected above the background fluorescence of the endogenous plant LGMs induced upon infection with the wild-type pathogen.

### Example 9

### Detecting Photon Emission from a Disease Organism Engineered to Express an Endogenous LGM (Embodiment II)—Integration of Photon Emission and Determination of Disease Organism Accumulation

The effectiveness of a plant treatment agent on a disease organism engineered to express an endogenous LGM and grown in liquid culture in wells of a microtitre plate is determined by integrating photon emission and determining the disease accumulation. Transgenic Magnaportha grisea strain MG619 engineered to express the ZsGreen FP as described above is cultured in liquid growth media with or without a plant treatment agent contained within wells of a 96 well microtitre plate in a manner similar to that described in Example 2.

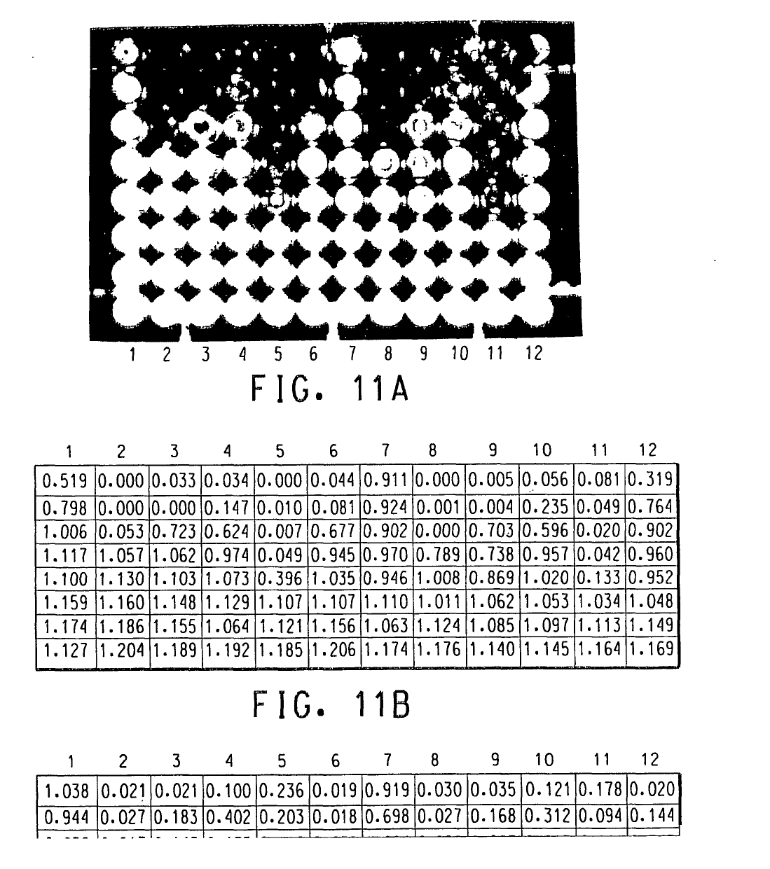

Plates to be rated for fungicide activity are first rated conventionally using a plate reader to measure optical density (OD) at 650 nm (see FIG. 11C). Reference and fluorescence image data is collected as described above using a Hamamatsu C2400 photon detector (see above), and processed for the photon emission from each well.

A grayscale image of the fluorescence data is presented in FIG. 11A. The normalized photon counts are represented in FIG. 11B. FIG. 11C represents the normalized OD data at 650 nm. Both the photon counts and OD 650 nm data have been normalized relative to the mean values for the respective measures for the controls in column 1, allowing easier comparison of the data from the two measurement types. Comparison of the photon counts and the OD measurements indicate that the methods yield comparable results.

1. A method for determining the effectiveness of a plant treatment agent in controlling a plant disease organism, comprising:
(a) applying the plant treatment agent to a substrate suitable for growth of the plant disease organism;
(b) inoculating the substrate with the disease organism; and either (A)
(A1c) after a period of time in which the organism can grow, applying a pro-light-generating moiety which is selectively transformed by the disease organism to a light-generating moiety;
(A1d) detecting photon emission from the light-generating moiety produced in (A1c); and
(A1e) determining the effectiveness of the plant treatment agent based on the amount of detected photon emission in (A1d); or (B) if the substrate is a plant substrate,
(B1c) after a period of time in which the disease can be manifested in the plant substrate, applying a pro-light-generating moiety which is selectively transformed by the disease organism or the plant substrate to a light-generating moiety;
(B1d) detecting photon emission from the light-generating moiety produced in (B1c) over an area of the plant substrate; and
(B1e) determining the effectiveness of the plant treatment agent based on the fraction of said plant substrate area from which photon emission is detected in (B1d); or (C) if the disease organism has an endogenous light-generating moiety that is not present in the substrate,
(C2c) after a period of time in which the organism can grow, detecting photon emission from the light-generating moiety; and
(C2d) determining the effectiveness of the plant treatment agent based on the detected photon emission in (C2c); or (D) if the substrate is a plant substrate having an endogenous light-generating moiety that is not present in the disease organism,
(D2c) after a period of time in which the disease can be manifested in the plant substrate, detecting photon emission from the light-generating moiety; and
(D2d) determining the effectiveness of the plant treatment agent based on the detected photon emission in (D2c).

2. A method for determining the effectiveness of a plant treatment agent in controlling a plant disease organism in accordance with claim 1, comprising
(a) applying the plant treatment agent to a substrate suitable for growth of the plant disease organism;
(b) inoculating the substrate with the disease organism;
(1c) after a period of time in which the organism can grow, applying a pro-light-generating moiety which is selectively transformed by the disease organism to a light-generating moiety;
(1d) detecting photon emission from the light-generating moiety; and
(1e) determining the effectiveness of the plant treatment agent based on the amount of detected photon emission.

3. The method of claim 2 wherein the disease organism is a fungal pathogen; and wherein the total amount of photon emission from a container with inoculated non-plant growth medium treated with a plant treatment agent is compared to the total photon emission from at least one untreated control, at least one standard and at least one blank.

4. A method for determining the effectiveness of a plant treatment agent in controlling a plant disease organism in accordance with claim 1, comprising
(a) applying the plant treatment agent to a plant substrate;
(b) inoculating the plant substrate with the disease organism;
(1c) after a period of time in which the disease can be manifested in the plant substrate, applying a pro-light-generating moiety which is selectively transformed by the disease organism or the plant substrate to a light-generating moiety;
(1d) detecting photon emission from the light-generating moiety over an area of the plant substrate; and
(1e) determining the effectiveness of the plant treatment agent based on the fraction of said plant substrate area from which photon emission is detected.

5. The method of claim 4 wherein the disease organism is a fungal pathogen; and wherein the pro-light generating moiety is selectively transformed by the fungal pathogen to a fluorescent light-generating moiety.

6. The method of claim 5 wherein the pro-light generating moiety is fluorescein diacetate and is sprayed onto the plant substrate.

7. The method of claim 4 wherein the pro-light generating moiety is selectively transformed by the plant.

8. A method for determining the effectiveness of a plant treatment agent in controlling a plant disease organism in accordance with claim 1, comprising
(a) applying the plant treatment agent to a substrate suitable for growth of the plant disease organism;
(b) inoculating the substrate with the disease organism having an endogenous light-generating moiety that is not present in the substrate;
(2c) after a period of time in which the organism can grow, detecting photon emission from the light-generating moiety; and
(2d) determining the effectiveness of the plant treatment agent based on the detected photon emission.

9. The method of claim 8 wherein the disease organism is a fungal pathogen; and wherein the total amount of photon emission from a container with inoculated non-plant growth medium treated with a plant treatment agent is compared to the total photon emission from at least one untreated control, at least one standard and at least one blank.

10. The method of claim 8 wherein the disease organism is a fungal pathogen that has an endogenous fluorescent light-generating moiety.

11. A method for determining the effectiveness of a plant treatment agent in controlling a plant disease organism in accordance with claim 1, comprising
(a) applying the plant treatment agent to a plant substrate;
(b) inoculating the plant substrate with the disease organism having an endogenous light-generating moiety that is not present in the plant substrate;
(2c) after a period of time in which the disease can be manifested in the plant substrate, detecting photon emission from the light-generating moiety; and
(2d) determining the effectiveness of the plant treatment agent based on the detected photon emission.

12. The method of claim 11 wherein the disease organism has an endogenous fluorescent light-generating moiety.

13. A method for determining the effectiveness of a plant treatment agent in controlling a plant disease organism in accordance with claim 1, comprising
(a) applying the plant treatment agent to a plant substrate having an endogenous light-generating moiety that is not present in the disease organism;
(b) inoculating the plant substrate with the disease organism;
(2c) after a period of time in which the disease can be manifested in the plant substrate, detecting photon emission from the light-generating moiety; and
(2d) determining the effectiveness of the plant treatment agent based on the detected photon emission.

14. The method of claim 13 wherein the plant substrate has an endogenous bioluminescent light-generating moiety.

15. The method of claim 13 wherein the plant substrate has an endogenous fluorescent light-generating moiety.

16. The method of any one of claims 11 through 15 wherein the effectiveness of the plant treatment agent is determined by measuring the amount of photon emission.

17. The method of any one of claims 11 through 15 wherein the effectiveness of the plant treatment agent is determined by measuring the fraction of the substrate area from which photon emission is detected.

18. The method of any one of claims 1 through 17 wherein the plant treatment agent is applied to the substrate (a) prior to inoculating (b) to determine the effectiveness of the plant treatment agent in preventing disease.

19. The method of any one of claims 1 through 17 wherein the substrate is inoculated (b) prior to the application of the plant treatment agent to the substrate (a) to determine the effectiveness of the plant treatment agent in curing disease.

20. A process for producing a crop protection agent that is suitable for controlling a plant disease caused by a plant disease organism and comprises a plant treatment agent, comprising:
determining the effectiveness of the plant treatment agent in controlling the plant disease organism using a method of any of claims 1 through 19.

21. A method for controlling a plant disease caused by a plant disease organism comprising:
determining that a plant treatment agent is effective in controlling the plant disease organism using a method of any of claims 1 through 19; and
applying to the plant or portion thereof to be protected, or to the plant seed or seedling to be protected, an effective amount of said plant treatment agent.
